(19) United States
(12) Patent Application Publication
(10) Pub. No.: US 2023/0024528 A1
(43) Pub. Date: Jan. 26, 2023

(54) METHODS OF USE OF ANTI-TREM2 ANTIBODIES

(71) Applicant: Alector LLC, South San Francisco, CA (US)

(72) Inventors: Tina SCHWABE, San Francisco, CA (US); Ilaria TASSI, South San Francisco, CA (US); Arnon ROSENTHAL, Woodside, CA (US); Hua LONG, San Carlos, CA (US); Santiago Viveros SALAZAR, South San Francisco, CA (US)

(73) Assignee: Alector LLC, South San Francisco, CA (US)

(21) Appl. No.: 17/782,127

(22) PCT Filed: Dec. 4, 2020
(86) PCT No.: PCT/US2020/063339

§ 371 Date: Jun. 2, 2022

Related U.S. Application Data
(60) Provisional application No. 63/005,110, filed on Apr. 3, 2020.
(60) Provisional application No. 62/944,298, filed on Dec. 5, 2019.

(51) Int. Cl. C07K 16/28; A61P 25/28

(52) U.S. Cl. CPC C07K 16/2803; A61P 25/28; A61K 2039/505

(57) ABSTRACT

The present disclosure is generally directed to the use of anti-TREM2 antibodies in preventing, reducing risk, or treating disease in an individual in need thereof.

### CROSS-REFERENCE TO RELATED APPLICATIONS

This application claims the benefit of U.S. Provisional Application No. 62/944,298, filed Dec. 5, 2019 and U.S. Provisional Application No. 63/005,110, filed Apr. 3, 2020, each of which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022003240SEQLIST.TXT, date recorded: Dec. 2, 2020, size: 88 KB).

### FIELD OF THE PRESENT DISCLOSURE

The present disclosure relates to therapeutic uses of anti-TREM2 antibodies.

### BACKGROUND OF THE PRESENT DISCLOSURE

Adult-onset leukoencephalopathy with axonal spheroids and pigmented glia (ALSP), pediatric-onset leukoencephalopathy are rare fatal neurological diseases which alter the “white matter” of the central nervous system in afflicted individuals (Freeman et al. (2009) “Adult onset leukodystrophy with neuroaxonal spheroids: Clinical, neuroimaging and neuropathologic observations.” Brain Pathol. 19(1): 39-47. PMID: 18422757; Rademakers et al. (2011) “Mutations in the colony stimulating factor 1 receptor (CSF1R) gene cause hereditary diffuse leukoencephalopathy with spheroids.” Nat Genet. 44(2):200-205. PMID: 22197934; Oosterhof et al. (2019) “Homozygous Mutations in CSF1R Cause a Pediatric-Onset Leukoencephalopathy and Can Result in Congenital Absence of Microglia.” Am J Hum Genet. 104(5):936-947. PMID: 30982608). Previously, ALSP was thought to be two separate conditions, hereditary diffuse leukoencephalopathy (HDLS) and familial pigmentary orthochromatic leukoencephalopathy (POLD). However, given patients with HDLS and POLD both can have pigmented glial cells and spheroids, HDLS and POLD are considered part of the same spectrum of disease encompassed by ALSP (Nicholson et al. (2013) “CSF1R mutations link POLD and HDLS as a single disease entity.” Neurology 80(11): 1033-1040. PMID: 23408870).

Patients with ALSP and pediatric-onset leukoencephalopathy characteristically have swelling in the axons of the brain, called spheroids. Recent research has linked mutations in the CSF1R gene to ALSP and pediatric-onset leukoencephalopathy (Rademakers et al. (2011); Nicholson et al. (2013); Oosterhof et al. (2019); Guo et al. (2019) “Bi-allelic CSF1R Mutations Cause Skeletal Dysplasia of Dysosteosclerosis-Pyle Disease Spectrum and Degenerative Encephalopathy with Brain Malformation.” Am J Hum Genet. 104(5):925-935. PMID: 30982609).

The human CSF1R gene encodes a protein called colony-stimulating factor 1 receptor (CSF1R). Colony-stimulating factor-1 (CSF-1), a homodimeric glycoprotein, is the primary ligand for CSF1R (Sherr et al. (1988) “Colony-stimulating factor-1 receptor (c-fms).” J Cell Biochem 38(3):179-187. PMID: 2852667). CSF1R is a type III tyrosine kinase growth factor receptor that belongs to the PDGF receptor family. Members of the receptor family have protein structures consisting of immunoglobulin-like domains, a transmembrane domain, and a protein kinase domain. Specifically, CSF1R is composed of a highly glycosylated extracellular ligand-binding domain, a transmembrane domain, and an intracellular protein tyrosine-kinase domain. CSF1R is found in the outer membrane of various cell types, including macrophages, and serves as the growth factor receptor for colony stimulating factor-1 (CSF-1) (Pridans et al. (2013) “CSF1R mutations in hereditary diffuse leukoencephalopathy with spheroids are loss of function.” Sci Rep 3: 3012. PMID: 24145216; Ridge et al. (1990) “FMS mutations in myelodysplastic, leukemic, and normal subjects.” 87(4): 1377-1380. PMID: 2406720; Oosterhof et al; Rademaker et al). Signaling via CSF1R has been shown to regulate the proliferation and development of macrophages, including microglial cells. Specifically, CSF1R signaling may be responsible for the generation of the majority of mature macrophages, including the microglia of the brain. CSF1R deficiency negatively impacts the development of microglia in the brain (Swerdlow et al. (2009) “Autosomal dominant subcortical gliosis presenting as frontotemporal dementia.” Neurology 72(3):260-267. PMID: 19153373; Baba et al. (2006) “Hereditary diffuse leukoencephalopathy with spheroids: clinical, pathologic and genetic studies of a new kindred.” Acta Neuropathol. 111(4):300-311. PMID: 16523341; Oosterhof et al.; Rademaker et al.; Guo et al., 2019).

At present, there are no effective treatment options for patients suffering from ALSP, pediatric-onset leukoencephalopathy, and related diseases. Available treatments manage disease symptoms rather than treating the disease. Thus, there is a need in the art for new treatments to provide therapeutic options and improve outcomes for patients afflicted by ALSP, pediatric-onset leukoencephalopathy, and related diseases.

All references cited herein, including patent applications and publications, are hereby incorporated by reference in their entirety.

### SUMMARY OF THE PRESENT DISCLOSURE

The present disclosure is generally directed to methods of treating individuals having a CSF1R-deficient disease comprising administering to the individual an antibody that binds to a TREM2 protein, where the antibody is an agonist.

Certain aspects of the present disclosure are based, at least in part, on the discovery that an agonistic anti-TREM2 antibody significantly improved viability of human macrophages that were grown in the presence of a CSF1R inhibitor, compared to human macrophages grown in the presence of a CSF1R inhibitor and a control IgG (see, e.g., Example 2).

Accordingly, In one aspect, the present disclosure provides a method of treating or preventing a CSF1R-deficient disease comprising administering to an individual in need thereof a therapeutically effective amount of an antibody that binds to a TREM2 protein, wherein the antibody is an agonist and wherein the antibody induces one or more TREM2 activities.

In some embodiments, the antibody enhances one or more TREM2 activities induced by binding of one or more TREM2 ligands to the TREM2 protein. In some embodiments, the antibody enhances the one or more TREM2 activities without blocking binding of the one or more TREM2 ligands to the TREM2 protein. In some embodiments, the antibody enhances binding of the one or more TREM2 ligands to the TREM2 protein. In some embodiments, the one or more TREM2 ligands are selected from the group consisting of E. coli cells, apoptotic cells, nucleic acids, anionic lipids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, lipidated amyloid beta peptide, and any combination thereof. In some embodiments, the antibody enhances the one or more TREM2 activities in the absence of cell surface clustering of TREM2. In some embodiments, the antibody enhances the one or more TREM2 activities by inducing or retaining cell surface clustering of TREM2.

In some embodiments, the TREM2 protein is a mammalian protein or a human protein. In some embodiments, the TREM2 protein is a wild-type protein, a naturally occurring variant, or a disease variant.

In some embodiments, the one or more TREM2 activities that are induced or enhanced by the antibody are selected from the group consisting of: (a) TREM2 binding to DAP12; (b) DAP12 phosphorylation; (c) activation of Syk kinase; (d) modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells; (e) recruitment of Syk to a DAP12/TREM2 complex; (f) increasing activity of one or more TREM2-dependent genes, optionally wherein the one or more TREM2-dependent genes comprise nuclear factor of activated T-cells (NFAT) transcription factors; (g) increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; (h) modulated expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells; (i) increasing memory; and (j) reducing cognitive deficit.

In some embodiments, the antibody promotes survival of macrophages cultured in the absence of CSF1. In some embodiments, the antibody decreases plasma levels of soluble TREM2 in vivo. In some embodiments, the antibody blocks cleavage of TREM2. In some embodiments, the antibody induces expression of CSF1R or increases levels of CSF1R in the individual compared to an untreated individual or an individual treated with a control antibody. In some embodiments, the induction of expression of CSF1R or the increase in level of CSF1R occurs in the brain of the individual. In some embodiments, the method comprises a step of measuring the level of CSF1R in a sample from the individual.

In some embodiments, the antibody is a murine antibody, a humanized antibody, a bispecific antibody, a multivalent antibody, a conjugated antibody, or a chimeric antibody. In some embodiments, the antibody is a monoclonal antibody.

In some embodiments, the antibody binds to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1.

In some embodiments, the antibody binds to one or more amino acid residues selected from the group consisting of D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1.

In some embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32).

In some embodiments, the antibody comprises a heavy chain variable region comprising the amino acid sequence of SEQ ID NO: 27 and a light chain variable region comprising the amino acid sequence of SEQ ID NO: 30.

In some embodiments, the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

In some embodiments, the antibody comprises a heavy chain variable region comprising the amino acid sequence of SEQ ID NO: 28 and a light chain variable region comprising the amino acid sequence of SEQ ID NO: 29.

In some embodiments, the antibody is a fragment and the fragment is an Fab, Fab′, Fab′-SH, F(ab′)2, Fv or scFv fragment. In some embodiments, the antibody is of the IgG class, the IgM class, or the IgA class. In some embodiments, the antibody is of the IgG class and has an IgG1, IgG2, IgG3, or IgG4 isotype. In some embodiments, the antibody has a human IgG1 isotype and comprises amino acid substitutions in the Fc region at the residue positions P331S and E430G, wherein the numbering of the residues is according to EU numbering.

In some embodiments, the antibody comprises: (a) a heavy chain comprising the amino acid of SEQ ID NO: 43, and a light chain comprising the amino acid sequence of SEQ ID NO: 47; or (b) a heavy chain comprising the amino acid of SEQ ID NO: 44, and a light chain comprising the amino acid sequence of SEQ ID NO: 47.

In some embodiments, the antibody comprises: (a) a heavy chain comprising the amino acid of SEQ ID NO: 45, and a light chain comprising the amino acid sequence of SEQ ID NO: 48; or (b) a heavy chain comprising the amino acid of SEQ ID NO: 46, and a light chain comprising the amino acid sequence of SEQ ID NO: 48.

In some embodiments, the individual is a human. In some embodiments, the CSF1R-deficient disease is adult-onset leukoencephalopathy with axonal spheroids and pigmented glia (ALSP). In some embodiments, the CSF1R-deficient disease is pediatric-onset leukoencephalopathy.

In some embodiments, the individual has a mutation in the CSF1R gene. In some embodiments, the mutation is in the portion of the CSFR1 gene encoding the intracellular protein tyrosine kinase domain. In some embodiments, the mutation is in any one of exons 11-21 of the CSFR1 gene. In some embodiments, the individual is heterozygous for the mutation in the CSFR1 gene. In some embodiments, the individual is homozygous for the mutation in the CSFR1 gene.

In some embodiments, the individual has or is at risk for a disease characteristic selected from the group consisting of leukoencephalopathy, axonal damage, axonal spheroids, myelin damage, loss of myelin sheaths, gliosis, autofluorescent lipid-laden macrophages, and axon destruction. In some embodiments, the individual has or is at risk for a symptom selected from the group consisting of abnormality of the cerebral white matter, behavioral changes, dementia, parkinsonism, seizures, motor aphasia, agraphia, acalculia, apraxia, bradykinesia, slow movements, central nervous system demyelination, depressivity, depression, frontal lobe dementia, gliosis, hyperreflexia, increased reflexes, extensor plantar response, hemiparesis, quadriparesis, leukoencephalopathy, memory impairment, forgetfulness, memory loss, memory problems, poor memory, mutism, inability to speak, muteness, neuronal loss in central nervous system, loss of brain cells, postural instability, balance impairment, rapid progressivity, rigidity, muscle rigidity, shuffling gait, shuffled walk, pyramidial signs, spasticity, involuntary muscle stiffness, involuntary muscle contraction, involuntary muscle spasms, personality problems, executive dysfunction.

In some embodiments, the individual has a disease selected from the group consisting of frontotemporal dementia (FTD), corticobasal syndrome (CBS), corticobasal degeneration (CBD), Alzheimer disease (AD), multiple sclerosis (MS), atypical cerebral autosomal dominant arteriopathy with subcortical infarcts and leukoencephalopathy (CADASIL), and Parkinson disease (PD).

In one aspect, the present disclosure provides a method of monitoring the treatment of an individual being administered an anti-TREM2 antibody comprising measuring the level of CSF1R in a sample from the individual before and after the individual has received one or more doses of an anti-TREM2 antibody. In some embodiments, the method comprises a step of assessing the activity of the anti-TREM2 antibody in the individual based on the level of CSF1R in the sample. In some embodiments, the sample is from the cerebrospinal fluid of the individual or the blood of the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows the effect of an anti-TREM2 agonistic antibody on human macrophage viability following withdrawal of M-CSF. Specifically, FIG. 1 shows the fold change in cell viability for human macrophages following withdrawal of M-CSF. Filled triangles represent human macrophages treated with M-CSF (50 ng/mL) alone, open triangles represent human macrophages treated with IgG1 (10 μg/mL), open circles represent human macrophages treated with AL2p-58 huIgG1 PSEG (1 μg/mL), and closed circles represent human macrophages treated with AL2p-58 huIgG1 PSEG (10 μg/mL). N=3 donors for each treatment; error bars represent standard error of the mean (SEM).

FIG. 2 shows the effect of an anti-TREM2 agonistic antibody on human macrophage viability following inhibition of the CSF1-Receptor (CSF1R). Specifically, FIG. 2 shows the fold change in cell viability for human macrophages treated with a CSF1R inhibitor (PLX3397). Closed triangles represent human macrophages treated with IgG1 (10 μg/mL) alone, open triangles represent human macrophages treated with PLX3397 (30 nM) alone, open circles represent human macrophages treated with AL2p-58 huIgG1 PSEG (10 μg/mL) alone, and filled circles represent human macrophages treated with both PLX3397 (30 nM) and AL2p-58 huIgG1 PSEG (10 μg/mL). N=3 donors for each treatment; error bars represent standard error of the mean (SEM).

FIG. 3 shows the effect of an anti-TREM2 agonistic antibody on expression of CSF1R protein in non-human primates. The change in CSF1R protein expression in samples from the frontal cortex following treatment with control IgG or with increasing concentrations of AL2p-58 huIgG1 PSEG is shown; p values were calculated with students t-test.

FIG. 4 shows the concentration of CSF1R protein in the frontal cortex and in the hippocampus of non-human primates administered AL2p-58 huIgG1 or control. The non-human primates were administered control or AL2p-58 huIgG1 intravenously once weekly for a total of 5 doses (N=5 per group). The concentrations of CSF1R protein (ng CSF1R protein/mg total protein) in the frontal cortex (left panel) and in the hippocampus (right panel) at 48 hours after the fifth administration of AL2p-58 huIgG1 or control are provided.

### DETAILED DESCRIPTION OF THE PRESENT DISCLOSURE

Provided herein are methods of treating disorders and diseases associated with deficiencies or other defects in CSF1R signaling by administering an agonist of TREM2. Such diseases or disorders include, but are not limited to, diseases associated with mutations in CSF1R, such as pediatric-onset leukoencephalopathy; adult-onset leukoencephalopathy with axonal spheroids and pigmented glia (ALSP); leukoencephalopathy, diffuse hereditary, with spheroids; adult-onset leukodystrophy with neuroaxonal spheroids; autosomal dominant leukoencephalopathy with neuroaxonal spheroids; hereditary diffuse leukoencephalopathy with axonal spheroids (HDLS); neuroaxonal leukodystrophy; pigmentary orthochromatic leukodystrophy; and familial pigmentary orthochromatic leukoencephalopathy (POLD). Agonists of TREM2 include anti-TREM2 antibodies that induce one or more TREM2 activities and/or enhance one or more activities induced by binding of one or more ligands to TREM2. For example, agonist anti-TREM2 antibodies may decrease soluble TREM2, induce spleen tyrosine kinase (Syk) phosphorylation, induce binding of TREM2 to DAP12, induce DAP12 phosphorylation, increase the proliferation, survival, and/or function of dendritic cells, macrophages, monocytes, osteoclasts, Langerhans cells of skin, Kupffer cells, and microglial cells (microglia), or increase the activity and/or expression of TREM2-dependent genes.

### Definitions

As used herein, the term “preventing” includes providing prophylaxis with respect to occurrence or recurrence of a particular disease, disorder, or condition, including delaying onset of a particular disease, disorder, or condition, in an individual that may be predisposed to, susceptible to, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. In some embodiments, the individual is human.

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an isolated anti-TREM2 antibody of the present disclosure, is one that has been identified, separated and/or recovered from a component of its production environment (e.g., naturally or recombinantly). Preferably, the isolated polypeptide is free from association with substantially all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain.

The “variable region” or “variable domain” of an antibody, such as an anti-TREM2 antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-TREM2 antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular cytotoxicity.

The term “monoclonal antibody” as used herein refers to an antibody, such as a monoclonal anti-TREM2 antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous in that they may be synthesized by a hybridoma culture, substantially uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3):253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J. Mol. Biol. 222:581-597 (1992); Sidhu et al., J. Mol. Biol. 338(2): 299-310 (2004); Lee et al., J. Mol. Biol. 340(5):1073-1093 (2004); Fellouse, Proc. Nat'l Acad. Sci. USA 101(34):12467-472 (2004); and Lee et al., J. Immunol. Methods 284(1-2):119-132 (2004), yeast presentation technologies (see, e.g., WO2009/036379A2; WO2010105256; WO2012009568, and Xu et al., Protein Eng. Des. Sel., 26(10): 663-70 (2013), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-TREM2 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-TREM2 antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments that are capable of binding and cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments may be produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which is recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) may have the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains, which enables the sFv to form the desired structure for antigen binding. For a review of the sFv, see Plückthun in The Pharmacology of Monoclonal Antibodies, vol. 113, Rosenburg and Moore eds., Springer-VerLAG-3, New York, pp. 269-315 (1994).

“Functional fragments” of antibodies, such as anti-TREM2 antibodies of the present disclosure, comprise a portion of an intact antibody, generally including the antigen binding or variable region of the intact antibody or the Fc region of an antibody which retains or has modified FcR binding capability.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody, such as a chimeric anti-TREM2 antibody of the present disclosure, in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies include antibodies in which the variable region of the antibody is derived from a murine antibody, and the constant region is derived from a human antibody. As used herein, “humanized antibody” is a subset of “chimeric antibodies.”

“Humanized” forms of non-human (e.g., murine) antibodies, such as humanized forms of anti-TREM2 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-TREM2 antibody of the present disclosure, that has been made using any of the techniques for making human antibodies as disclosed herein or otherwise known in the art. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology. Alternatively, human antibodies can also be prepared by employing yeast libraries and methods as disclosed in, for example, WO2009/036379A2; WO2010105256; WO2012009568; and Xu et al., Protein Eng. Des. Sel., 26(10): 663-70 (2013).

The term “hypervariable region” or “HVR” when used herein refers to the regions of an antibody-variable domain, such as that of an anti-TREM2 antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable domain residues are numbered according to Kabat et al., supra, for each of these extended HVR definitions.

“Framework” or “FR” residues are those variable domain residues other than the HVR residues as herein defined.

The phrase “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system,” “EU numbering” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. References to residue numbers in the variable domain of antibodies means residue numbering by the Kabat numbering system. References to residue numbers in the constant domain of antibodies means residue numbering by the EU numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferably those changes occur at only three, two, or one of positions 71H, 73H and 78H; for instance, the amino acid residues at those positions may by 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, Md. (1991). For the VL, the subgroup may be, e.g., subgroup kappa I, kappa II, kappa III or kappa IV as in Kabat et al., supra. Additionally, for the VH, the subgroup may be, e.g., subgroup I, subgroup II, or subgroup III as in Kabat et al., supra.

An “amino acid modification” at a specified position, e.g., of an anti-TREM2 antibody of the present disclosure, refers to the substitution or deletion of the specified residue, or the insertion of at least one amino acid residue adjacent to the specified residue. Insertion “adjacent” to a specified residue means insertion within one to two residues thereof. The insertion may be N-terminal or C-terminal to the specified residue. The preferred amino acid modification herein is a substitution.

An “affinity matured” antibody, such as an affinity matured anti-TREM2 antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity matured antibodies may be produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As use herein, the term “specifically binds” refers to measurable and reproducible binding interactions between a target and an antibody, such as between an anti-TREM2 antibody and TREM2, that is determinative of the presence of the target within a heterogeneous population of molecules, such as biological molecules. For example, an antibody, such as an anti-TREM2 antibody of the present disclosure, that specifically binds to a target or an epitope of the target is an antibody that preferentially binds this target or epitope, e.g., with greater affinity or avidity, than it binds to other unrelated targets or epitopes. It is also understood that an antibody that specifically binds to a first target may or may not specifically bind to a second target. As such, “specific binding” does not necessarily require (although it can include) exclusive binding. An antibody that specifically binds to a target may have an association constant of at least about 10³ M⁻¹ or 10⁴ M⁻¹, sometimes about 10 s M⁻¹ or 10⁶ M⁻⁴, in other instances about 10⁶ M⁻¹ or 10⁷ M⁻¹, about 10⁸ M⁻⁴ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, or Vashist and Luong (2018) Handbook of Immunoassay Technologies, Approaches, Performances, and Applications, Academic Press, for a description of immunoassay formats and conditions that can be used to determine specific immunoreactivity.

As used herein, an antibody “inhibits interaction” between two proteins when the antibody disrupts, reduces, or completely eliminates an interaction between the two proteins by binding to one of the two proteins.

An “agonist” antibody is an antibody that induces (e.g., increases) one or more activities or functions of a target upon binding to the target.

An “antagonist” antibody or a “blocking” antibody is an antibody that reduces or eliminates (e.g., decreases) antigen binding to one or more binding partners after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more binding partners and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region, e.g. from about one to about ten amino acid substitutions, and preferably from about one to about five amino acid substitutions in a native sequence Fc region. The variant Fc region herein will preferably possess at least about 80% homology with a native sequence Fc region, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126: 330-41 (1995). Other FcRs are encompassed by the term “FcR” herein. FcRs can also increase the serum half-life of antibodies.

Binding to FcR in vivo and serum half-life of human FcR high-affinity binding polypeptides can be assayed, e.g., in transgenic mice or transfected human cell lines expressing human FcR, or in primates to which the polypeptides having a variant Fc region are administered. WO 2004/42072 (Presta) describes antibody variants with improved or diminished binding to FcRs. See also, e.g., Shields et al., J. Biol. Chem. 9(2):6591-6604 (2001).

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule, e.g., encoding an antibody such as an anti-TREM2 antibody of the present disclosure, is a nucleic acid molecule that is identified and separated from at least one contaminant nucleic acid molecule with which it is ordinarily associated in the environment in which it was produced. Preferably, the isolated nucleic acid is free of association with substantially all components associated with the production environment. The isolated nucleic acid molecules encoding the polypeptides and antibodies herein are distinguished from nucleic acid existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps”; substitution of one or more of the naturally occurring nucleotides with an analog; and internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can contain or contains a vector(s) or other exogenous nucleic acid, e.g., that incorporates a polynucleotide insert(s). In some embodiments, the vector or other exogenous nucleic acid is incorporated into the genome of the host cell. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspect and embodiments of the present disclosure described herein include “comprising,” “consisting,” and “consisting essentially of” aspects and embodiments.

### METHODS OF THE PRESENT DISCLOSURE

The present disclosure provides methods of treating, preventing, or reducing risk in an individual having a CSF1R-deficient disease comprising administering to the individual in need thereof a therapeutically effective amount of an antibody that binds to a TREM2 protein, where the antibody is an agonist.

“CSF1R-deficient disease,” as used herein, refers to any disease, disorder or condition resulting from deficiencies or other defects in CSF1R signaling. In certain embodiments, the disease, disorder or condition involves a CSF1R protein with reduced function as compared to CSF1R protein considered to have “wild type” function or that has function considered to be within normal range. In some embodiments, CSF1R-deficient diseases are characterized by a mutation in the CSF1R gene in the affected individual. The mutation typically results in reduced function of CSF1R in the affected individual. The mutation may be of any type, including, for example, a missense mutation, an indel, or a mutation generating a truncated protein product.

Without wishing to be bound by theory, it is believed that agonizing TREM2 will ameliorate the effects of CSF1R deficiency in an individual having a CSF1R-deficient disease. In certain embodiments, a TREM2 antibody as described herein may activate or increase signaling downstream of CSF1R to compensate for or otherwise rescue a CSF1R deficiency. In certain embodiments, a TREM2 antibody as described herein may induce or increase expression of CSF1R that is deficient in signaling, wherein increasing the amount of such CSF1R results in a sufficient amount of signaling.

CSF1R-deficient diseases include, without limitation, pediatric-onset leukoencephalopathy and adult-onset leukoencephalopathy with axonal spheroids and pigmented glia (ALSP). In some embodiments, the CSF1R-deficient disease is ALSP. In some embodiments, the CSF1R-deficient disease is pediatric-onset leukoencephalopathy.

Adult-Onset Leukoencephalopathy with Axonal Spheroids and Pigmented Glia

In some embodiments, the present disclosure provides methods of treating, preventing, or reducing risk in an individual having ALSP comprising administering to the individual in need thereof a therapeutically effective amount of an antibody that binds to a TREM2 protein, where the antibody is an agonist.

Adult-onset leukoencephalopathy with axonal spheroids and pigmented glia (ALSP) is an autosomal dominant neurological condition characterized by changes in specific regions of the brain. ALSP is caused by a mutation in the gene encoding CSF1R. “Leukoencephalopathy,” damage to the white matter of the brain, is a classic characteristic of ALSP. In addition, axonal damage caused by swelling called spheroids, is another common characteristic of ALSP. Additional common ALSP disease characteristics include myelin damage, loss of myelin sheaths, gliosis, autofluorescent lipid-laden macrophages, and axon destruction. Further, it is believed that damage to myelin and axons contributes to the numerous neurological signs and symptoms of individuals afflicted with ALSP (Oosterhof et al; Rademaker et al, Guo et al, 2019).

Common symptoms of ALSP include, but are not limited to, abnormality of the cerebral white matter, behavioral changes, dementia, parkinsonism, seizures, motor aphasia, agraphia, acalculia, apraxia, bradykinesia, slow movements, central nervous system demyelination, depressivity, depression, frontal lobe dementia, gliosis, hyperreflexia, increased reflexes, extensor plantar response, hemiparesis, quadriparesis, leukoencephalopathy, memory impairment, forgetfulness, memory loss, memory problems, poor memory, mutism, inability to speak, muteness, neuronal loss in central nervous system, loss of brain cells, postural instability, balance impairment, rapid progressivity, rigidity, muscle rigidity, shuffling gait, shuffled walk, pyramidial signs, spasticity, involuntary muscle stiffness, involuntary muscle contraction, involuntary muscle spasms, personality problems, and executive dysfunction.

Previously, the disease now referred to as ALSP was thought to be two different diseases, hereditary diffuse leukoencephalopathy with axonal spheroids (HDLS) and familial pigmentary orthochromatic leukoencephalopathy (POLD). Indeed, clinicians believed that pigmented glial cells were a feature of POLD, but not HDLS. Further, clinicians believed that spheroids were a feature of HDLS, but not POLD. However, a detailed analysis of the clinical and pathological features of each disease demonstrated that patients with HDLS and POLD presented with pigmented glial cells and spheroids. As such, HDLS and POLD are now considered part of the same spectrum of diseases encompassed by ALSP (Nicholson et al. (2013)).

Exemplary alternative names for ALSP include, without limitation, leukoencephalopathy, diffuse hereditary, with spheroids; adult-onset leukodystrophy with neuroaxonal spheroids; autosomal dominant leukoencephalopathy with neuroaxonal spheroids; hereditary diffuse leukoencephalopathy with axonal spheroids (HDLS); neuroaxonal leukodystrophy; pigmentary orthochromatic leukodystrophy; and familial pigmentary orthochromatic leukoencephalopathy (POLD).

In some embodiments, administering an anti-TREM2 agonist antibody can prevent, reduce the risk, and/or treat adult-onset leukoencephalopathy with axonal spheroids and pigmented glia (ALSP). In some embodiments, administering an anti-TREM2 antibody induces one or more TREM2 activities in an individual having adult-onset leukoencephalopathy with axonal spheroids and pigmented glia (ALSP). Without wishing to be bound by theory, it is believed that agonizing TREM2 will reduce the effects of CSF1R deficiency in an individual having ALSP.

Pediatric-Onset Leukoencephalopathy

Pediatric-onset leukoencephalopathy is a rare fatal neurological disease caused by mutations in the CSF1R gene. Pediatric-onset leukoencephalopathy is characterized by distinct pediatric phenotypes including: 1) developmental regression, 2) trouble with motor skills at onset, 3) epilepsy, and 4) cognitive decline. Recent investigations demonstrate that, as compared to ALSP, which is caused by heterozygous mutations in the CSF1R gene, individuals afflicted with pediatric-onset leukoencephalopathy have homozygous mutations in the CSF1R gene. Further, while individuals with pediatric-onset leukoencephalopathy have many neuroimaging features that overlap with ALSP, they also have distinct neuroimaging features that are not present in individuals with ALSP (Oosterhof et al. (2019)).

In some embodiments, administering an anti-TREM2 agonist antibody can prevent, reduce the risk, and/or treat pediatric-onset leukoencephalopathy. In some embodiments, administering an anti-TREM2 antibody induces one or more TREM2 activities in an individual having pediatric-onset leukoencephalopathy. Without wishing to be bound by theory, it is believed that agonizing TREM2 will reduce the effects of CSF1R deficiency in an individual having pediatric-onset leukoencephalopathy.

CSF1R Mutations

In some embodiments, the individual having the CSF1R-deficient disease has a mutation in the CSF1R gene. As detailed above, the human CSF1R gene encodes colony stimulating factor 1 receptor (CSF1R). CSF1R may also be referred to by one of the following names: C-FMS, CD115, CD115 antigen, CSF-1 receptor, CSF-1R, CSF1R_HUMAN, CSFR, FIM2, FMS, FMSproto-oncogene, CSF-1-R, M-CSF-R, BANDDOS, v-FMS, and c-FMSproto-oncogene. The chromosomal location of the CSF1R gene is 5q32 and its molecular location is base pairs 150,053,291 to 150,113,372 on chromosome 5. Further, the GenBank identifier for the CSF1R gene is NG_012303.

The CSF1R protein is a type III tyrosine kinase growth factor receptor that belongs to the PDGF receptor family. Specifically, CSF1R is composed of a highly glycosylated extracellular ligand-binding domain, a transmembrane domain, and an intracellular protein tyrosine kinase domain. CSF1R is a cell surface receptor that acts as the receptor primarily for CSF-1, a cytokine that regulates the survival, proliferation, differentiation, and function of mononuclear phagocytic cells, including microglia. Binding of CSF-1 to CSF1R results in the formation of receptor homodimers and subsequent auto-phosphorylation of several tyrosine residues in the cytoplasmic domain.

Any sequencing method known in the art may be used to identify mutations in the CSF1R gene. For example, a non-exhaustive list of sequencing methods that can be used to identify CSF1R mutations includes Sanger sequencing, whole exome sequencing, and next generation sequencing.

In some embodiments, the mutation in the CSF1R gene is in the portion of the gene encoding the intracellular protein tyrosine kinase domain. In some embodiments, the mutation in the CSF1R gene is in any one of exons 11-21. In some embodiments, an individual with a CSF1R-deficient disease is heterozygous for the mutation in the CSF1R gene. In some embodiments, an individual with a CSF1R-deficient disease is homozygous for the mutation in the CSF1R gene. In some embodiments, the mutation in the CSF1R gene is in the portion of the gene encoding an immunoglobulin-like domain. In some embodiments, the mutation in the CSF1R gene is in the portion of the gene encoding the transmembrane domain. In some embodiments, the mutation in the CSF1R gene is in the portion of the gene encoding the regulatory juxtamembrane domain.

Exemplary mutations in the CSF1R gene include, without limitation, c.1754-2A>G (p.G585_K619delinsA), c.1766G>A (p.G589E), c1897G>A (p.E633K), c.2297T>C (p.M766T), c.2308G>C (p.A770P), c.2320-2A>G (pC774 N814del), c.2324T>A (p.1775N), c.2381T>C (p.1794T), c.2442+5G>C (p.C774_N814delinsQGLQSHVGPSLPSSSPQAQ), c.2509G>T (p.D837Y), c.2546_2548delTCT (p.F849del), c.2546T>C (p.F849S), c.2603T>C (p.L868P), c.2624T>C (p.M875T), and c.2632C>A (p.P878T) (Oosterhof et al; Rademaker et al.).

In some embodiments, administering an anti-TREM2 antibody of the present disclosure can prevent, reduce the risk, and/or treat a CSF1R-deficient disease caused by a mutant variant of CSF1R. In some embodiments, administering an anti-TREM2 antibody may induce one or more TREM2 activities in an individual having a CSF1R-deficient disease caused by a mutant variant of CSF1R.

Comorbidities

Individuals with CSF1R-deficient diseases may suffer from and/or have been diagnosed with additional diseases, such as, for example, frontotemporal dementia (FTD), corticobasal syndrome (CBS), corticobasal degeneration (CBD), Alzheimer disease (AD), multiple sclerosis (MS), atypical cerebral autosomal dominant arteriopathy with subcortical infarcts and leukoencephalopathy (CADASIL), and Parkinson disease (PD).

### TREM2 Proteins

The present disclosure provides methods of treating, preventing, or reducing risk in an individual having a CSF1R-deficient disease comprising administering to the individual an antibody that binds to a TREM2 protein wherein the antibody is an agonist.

Triggering receptor expressed on myeloid cells-2 (TREM2) is variously referred to as TREM-2, TREM2a, TREM2b, TREM2c, triggering receptor expressed on myeloid cells-2a, and triggering receptor expressed on monocytes-2. TREM2 is a 230 amino acid membrane protein. TREM2 is an immunoglobulin-like receptor primarily expressed on myeloid lineage cells, including without limitation, macrophages, dendritic cells, monocytes, Langerhans cells of skin, Kupffer cells, osteoclasts, and microglia. In some embodiments, TREM2 forms a receptor signaling complex with DAP12. In some embodiments, TREM2 phosphorylates and signals through DAP12 (an ITAM domain adaptor protein). In some embodiments TREM2 signaling results in the downstream activation of PI3K or other intracellular signals. On myeloid cells, Toll-like receptor (TLR) signals are important for the activation of TREM2 activities, e.g., in the context of an infection response. TLRs also play a key role in the pathological inflammatory response, e.g., TLRs expressed in macrophages and dendritic cells.

TREM2 proteins of the present disclosure include, without limitation, a human TREM2 protein (Uniprot Accession No. Q9NZC2; SEQ ID NO: 1), and a non-human mammalian TREM2 protein, such as mouse TREM2 protein (Uniprot Accession No. Q99NH8; SEQ ID NO: 2), rat TREM2 protein (Uniprot Accession No. D3ZZ89; SEQ ID NO: 3), Rhesus monkey TREM2 protein (Uniprot Accession No. F6QVF2; SEQ ID NO: 4), cynomolgus monkey TREM2 protein (NCBI Accession No. XP_015304909.1; SEQ ID NO: 5), equine TREM2 protein (Uniprot Accession No. F7D6L0; SEQ ID NO: 6), pig TREM2 protein (Uniprot Accession No. H2EZZ3; SEQ ID NO: 7), and dog TREM2 protein (Uniprot Accession No. E2RP46; SEQ ID NO: 8). As used herein “TREM2 protein” refers to both wild-type sequences and naturally occurring variant sequences.

In some embodiments, an example of a human TREM2 amino acid sequence is set forth below as SEQ ID NO: 1:

```
                10 20 30 40
MEPLRLLILL FVTELSGAHN TTVFQGVAGQ SLQVSCPYDS
                50 60 70 80
MKHWGRRKAW CRQLGEKGPC QRVVSTHNLW LLSFLRRWNG
                90 100 110 120
STAITDDTLG GTLTITLRNL QPHDAGLYQC QSLHGSEADT
              130 140 150 160
LRKVLVEVLA DPLDHRDAGD LWFPGESESF EDAHVEHSIS
              170 180 190 200
RSLLEGEIPF PPTSILLLLA CIFLIKILAA SALWAAAWHG
              210 220 230
QKPGTHPPSE LDCGHDPGYQ LQTLPGLRDT
```

In some embodiments, the human TREM2 is a preprotein that includes a signal peptide. In some embodiments, the human TREM2 is a mature protein. In some embodiments, the mature TREM2 protein does not include a signal peptide. In some embodiments, the mature TREM2 protein is expressed on a cell. In some embodiments, TREM2 contains a signal peptide located at amino acid residues 1-18 of human TREM2 (SEQ ID NO: 1); an extracellular immunoglobulin-like variable-type (IgV) domain located at amino acid residues 29-112 of human TREM2 (SEQ ID NO: 1); additional extracellular sequences located at amino acid residues 113-174 of human TREM2 (SEQ ID NO: 1); a transmembrane domain located at amino acid residues 175-195 of human TREM2 (SEQ ID NO: 1); and an intracellular domain located at amino acid residues 196-230 of human TREM2 (SEQ ID NO: 1). The TREM2 cleavage site has been identified as occurring on the C-terminal side of Histidine 157 (see WO2018/015573), and cleavage at that site leads to shedding of the relevant portion of the TREM2 extracellular domain, detectable as an increase in soluble TREM2 (sTREM2) corresponding to that portion of TREM2.

The transmembrane domain of human TREM2 contains a lysine at amino acid residue 186 that can interact with an aspartic acid in DAP12, which is a key adaptor protein that transduces signaling from TREM2, TREM1, and other related IgV family members.

### Anti-TREM2 Antibodies

Certain aspects of the present disclosure relate to antibodies (e.g., monoclonal antibodies) that bind to a TREM2 protein, where the anti-TREM2 antibody is an agonist. In some embodiments, antibodies of the present disclosure bind a mature TREM2 protein. In some embodiments, antibodies of the present disclosure bind a mature TREM2 protein, wherein the mature TREM2 protein is expressed on a cell. In some embodiments, antibodies of the present disclosure bind a TREM2 protein expressed on one or more human cells selected from human dendritic cells, human macrophages, human monocytes, human osteoclasts, human Langerhans cells of skin, human Kupffer cells, human microglia, and any combinations thereof.

Anti-TREM2 Antibodies that Induce Activity and/or Enhance Ligand-Induced Activity

In some embodiments, an anti-TREM2 antibody of the present disclosure is an agonist antibody that induces one or more TREM2 activities. In some embodiments the antibody induces one or more activities of TREM2 after binding to a TREM2 protein that is expressed on a cell.

In some embodiments, anti-TREM2 antibodies of the present disclosure bind to a TREM2 protein without competing with, inhibiting, or otherwise blocking one or more TREM2 ligands from binding to the TREM2 protein. Examples of TREM2 ligands include, without limitation, TREM2 ligands expressed by E. coli cells, apoptotic cells, nucleic acids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, and lipidated amyloid beta peptide. Accordingly, in certain embodiments, the one or more TREM2 ligands comprise E. coli cells, apoptotic cells, nucleic acids, anionic lipids, zwitterionic lipids, negatively charged phospholipids, phosphatidylserine (PS), sulfatides, phosphatidylcholin, sphingomyelin (SM), phospholipids, lipidated proteins, proteolipids, lipidated peptides, and lipidated amyloid beta peptide.

Anti-TREM2 antibodies used in the methods of the present disclosure are agonist antibodies. In some embodiments, antibodies of the present disclosure that bind a TREM2 protein may include agonist antibodies that due to their epitope specificity bind TREM2 and activate one or more TREM2 activities. In some embodiments, such antibodies may bind to the ligand-binding site on TREM2 and mimic the action of one or more TREM2 ligands, or stimulate TREM2 to transduce signal by binding to one or more domains that are not the ligand-binding sites. In some embodiments, the antibodies do not compete with or otherwise block ligand binding to TREM2. In some embodiments, the antibodies, act additively or synergistically with one or more TREM2 ligands to activate and/or enhance one more TREM2 activities, as set forth below.

Agonist anti-TREM2 antibodies of the present disclosure may display the ability to bind TREM2 without blocking simultaneous binding of one or more TREM2 ligands. The anti-TREM2 antibodies of the present disclosure may further display additive and/or synergistic functional interactions with one or more TREM2 ligands. Thus, in some embodiments, the maximal activity of TREM2 when bound to anti-TREM2 antibodies of the present disclosure in combination with one or more TREM2 ligands of the present disclosure may be greater (e.g., enhanced) than the maximal activity of TREM2 when exposed to saturating concentrations of ligand alone or to saturating concentrations of the antibody alone. In addition, the activity of TREM2 at a given concentration of TREM2 ligand may be greater (e.g., enhanced) in the presence of the antibody.

Accordingly, in some embodiments, anti-TREM2 antibodies of the present disclosure have an additive effect with the one or more TREM2 ligands to enhance the one or more TREM2 activities when bound to the TREM2 protein. In some embodiments, anti-TREM2 antibodies of the present disclosure synergize with the one or more TREM2 ligands to enhance the one or more TREM2 activities. In some embodiments, anti-TREM2 antibodies of the present disclosure increase the potency of the one or more TREM2 ligands to induce the one or more TREM2 activities, as compared to the potency of the one or more TREM2 ligands to induce the one or more TREM2 activities in the absence of the antibody. In some embodiments, anti-TREM2 antibodies of the present disclosure enhance the one or more TREM2 activities in the absence of cell surface clustering of TREM2. In some embodiments, anti-TREM2 antibodies of the present disclosure enhance the one or more TREM2 activities by inducing or retaining cell surface clustering of TREM2. In some embodiments, anti-TREM2 antibodies of the present disclosure are clustered by one or more Fc-gamma receptors expressed on one or more immune cells, including without limitation, B cells and microglial cells. In some embodiments, enhancement of the one or more TREM2 activities induced by binding of one or more TREM2 ligands to the TREM2 protein is measured on primary cells, including without limitation, dendritic cells, bone marrow-derived dendritic cells, monocytes, microglia, macrophages, neutrophils, NK cells, osteoclasts, Langerhans cells of skin, and Kupffer cells, or on cell lines.

In certain embodiments, an anti-TREM2 antibody of the present disclosure that enhances one or more TREM2 activities induced by binding of one or more TREM2 ligands to the TREM2 protein induces at least a 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater increase in the one or more TREM2 activities as compared to levels of the one or more TREM2 activities induced by binding of the one or more TREM2 ligands to the TREM2 protein in the absence of the anti-TREM2 antibody.

In some embodiments, TREM2 activities that may be induced and/or enhanced by anti-TREM2 antibodies of the present disclosure and/or one or more TREM2 ligands of the present disclosure include, without limitation, TREM2 binding to DAP12; DAP12 phosphorylation; activation of Syk kinase; modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; increasing memory; and reducing cognitive deficit. In some embodiments, anti-TREM2 antibodies of the present disclosure increase memory and/or reduce cognitive deficit when administered to an individual.

Syk Phosphorylation

In some embodiments, the anti-TREM2 antibodies of the present disclosure may induce spleen tyrosine kinase (Syk) phosphorylation after binding to a TREM2 protein expressed in a cell.

Spleen tyrosine kinase (Syk) is an intracellular signaling molecule that functions downstream of TREM2 by phosphorylating several substrates, thereby facilitating the formation of a signaling complex leading to cellular activation and inflammatory processes.

In some embodiments, the ability of agonist TREM2 antibodies to induce Syk activation is determined by culturing mouse macrophages and measuring the phosphorylation state of Syk protein in cell extracts. In some embodiments, bone marrow-derived macrophages (BMDM) from wild-type (WT) mice, from TREM2 knockout (KO) mice, and from mice that lack expression of functional Fc receptor common gamma chain gene (FcgR KO; REF: Takai T 1994. Cell 76(3):519-29) are starved for 4 hours in 1% serum RPMI and then removed from tissue culture dishes with PBS-EDTA, washed with PBS, and counted. In some embodiments, the cells are coated with full-length TREM2 antibodies, or with control antibodies for 15 minutes on ice. In some embodiments, after washing with cold PBS, cells are incubated at 37° C. for the indicated period of time in the presence of goat anti-human IgG. In some embodiments, after stimulation, cells are lysed with lysis buffer (1% v/v NP-40%, 50 Mm Tris-HCl (pH 8.0), 150 mM NaCl, 1 mM EDTA, 1.5 mM MgCl₂, 10% glycerol, plus protease and phosphatase inhibitors) followed by centrifugation at 16,000 g for 10 min at 4° C. to remove insoluble materials. In some embodiments, lysates are then immunoprecipitated with anti-Syk antibody (N-19 for BMDM or 4D10 for human DCs, Santa Cruz Biotechnology). In some embodiments, precipitated proteins are fractionated by SDS-PAGE, transferred to PVDF membranes and probed with anti-phosphotyrosine antibody (4G10, Millipore). In some embodiments, to confirm that all substrates are adequately immunoprecipitated, immunoblots are reprobed with anti-Syk antibody (Abcam, for BMDM) or anti-Syk (Novus Biological, for human DCs). In some embodiments, visualization is performed with the enhanced chemiluminescence (ECL) system (GE healthcare), as described (e.g., Peng et al., (2010) Sci Signal., 3(122): ra38).

DAP12 Binding and Phosphorylation

In some embodiments, the anti-TREM2 antibodies of the present disclosure may induce binding of TREM2 to DAP12. In other embodiments, the anti-TREM2 antibodies of the present disclosure may induce DAP12 phosphorylation after binding to a TREM2 protein expressed in a cell. In other embodiments, TREM2-mediated DAP12 phosphorylation is induced by one or more SRC family tyrosine kinases. Examples of Src family tyrosine kinases include, without limitation, Src, Syk, Yes, Fyn, Fgr, Lck, Hck, Blk, Lyn, and Frk.

DAP12 is variously referred to as TYRO protein tyrosine kinase-binding protein, TYROBP, KARAP, and PLOSL. DAP12 is a transmembrane signaling protein that contains an immunoreceptor tyrosine-based activation motif (ITAM) in its cytoplasmic domain. In certain embodiments, the anti-TREM2 antibody may induce DAP12 phosphorylation in its ITAM motif. Any method known in the art for determining protein phosphorylation, such as DAP12 phosphorylation, may be used.

In some embodiments, DAP12 is phosphorylated by SRC family kinases, resulting in the recruitment and activation of the Syk kinase, ZAP70 kinase, or both, to a DAP12/TREM2 complex.

In some embodiments, the ability of TREM2 antibodies to induce DAP12 activation is determined by culturing mouse macrophages and measuring the phosphorylation state of DAP12 protein in cell extracts. In some embodiments, before stimulation with antibodies, mouse wild-type (WT) bone marrow-derived macrophages (BMDM) and TREM2 knockout (KO) BMDM are starved for 4 h in 1% serum RPMI. In some embodiments, 15×10⁶ cells are incubated in ice for 15 min with full-length TREM2 antibodies or control antibodies. In some embodiments, cells are washed and incubated at 37° C. for the indicated period of time in the presence of goat anti-human IgG. In some embodiments, after stimulation, cells are lysed with lysis buffer (1% v/v n-Dodecyl-β-D-maltoside, 50 Mm Tris-HCl (pH 8.0), 150 mM NaCl, 1 mM EDTA, 1.5 mM MgCl₂, 10% glycerol, plus protease and phosphatase inhibitors), followed by centrifugation at 16,000 g for 10 min at 4° C. to remove insoluble materials. In some embodiments, cell lysate is immunoprecipitated with a second TREM2 antibody (R&D Systems). In some embodiments, precipitated proteins are fractionated by SDS-PAGE, transferred to PVDF membranes, and probed with anti-phosphotyrosine Ab (4G10, Millipore). In some embodiments, the membrane is stripped and reprobed with anti-DAP12 antibody (Cells Signaling, D7G1X). In some embodiments, each cell lysate used for TREM2 immunoprecipitations contains an equal amount of proteins, as indicated by a control antibody (anti-Actin, Santa Cruz).

Proliferation, Survival and Functionality of TREM2-Expressing Cells

In some embodiments, the anti-TREM2 antibodies of the present disclosure may increase the proliferation, survival, and/or function of dendritic cells, macrophages, monocytes, osteoclasts, Langerhans cells of skin, Kupffer cells, and microglial cells (microglia) after binding to TREM2 protein expressed in a cell. In some embodiments, the anti-TREM2 antibodies of the present disclosure do not inhibit the growth (e.g., proliferation and/or survival) of one or more innate immune cells.

Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most infections from reaching the vulnerable nervous tissue. In the case where infectious agents are directly introduced to the brain or cross the blood-brain barrier, microglial cells must react quickly to decrease inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T-cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

As used herein, macrophages of the present disclosure include, without limitation, M1 macrophages, activated M1 macrophages, and M2 macrophages. As used herein, microglial cells of the present disclosure include, without limitation, M1 microglial cells, activated M1 microglial cells, and M2 microglial cells.

In some embodiments, anti-TREM2 antibodies of the present disclosure may increase the expression of CD83 and/or CD86 on dendritic cells, monocytes, and/or macrophages.

As used herein, the rate of proliferation, survival, and/or function of macrophages, dendritic cells, monocytes, and/or microglia may include increased expression if the rate of proliferation, survival, and/or function of dendritic cells, macrophages, monocytes, osteoclasts, Langerhans cells of skin, Kupffer cells, and/or microglia in a subject treated with an anti-TREM2 antibody of the present disclosure is greater than the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In some embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody. In other embodiments, an anti-TREM2 antibody of the present disclosure may increase the rate of proliferation, survival, and/or function of dendritic cells, macrophages, monocytes, osteoclasts, Langerhans cells of skin, Kupffer cells, and/or microglia in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the rate of proliferation, survival, and/or function of dendritic cells, macrophages, monocytes, osteoclasts, Langerhans cells of skin, Kupffer cells, and/or microglia in a corresponding subject that is not treated with the anti-TREM2 antibody.

In some embodiments, to evaluate the ability of anti-TREM2 antibodies to induce or enhance cell survival in vitro, macrophages deficient in the gamma chain subunit of FcgRI, FcgRIII, and FceRI receptors (Fcgr1KO mice, RET: Takai T, Li M, Sylvestre D, Clynes R, Ravetch J. (1994). Cell, 76:519-529) are cultured in the presence of plate-bound anti-TREM2 antibodies and cell viability is determined when cells are cultured in suboptimal growth conditions. In some embodiments, murine bone marrow precursor cells from FcgR1 KO mice (Taconic, Model 584) are obtained by flushing tibial and femoral marrow cells with cold PBS. In some embodiments, after one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. In some embodiments, to analyze cell viability of bone marrow-derived macrophages, cells are prepared as above and plated at 2.5×10⁴/200 μl in a 96-well plate with suboptimal amounts of M-CSF (10 ng/ml) in non-tissue culture treated plates for two days. In some embodiments, cells are then quantified using the ToxGlo™ kit (Promega) and luminescence is determined as a measure of cell viability. In some embodiments, all experiments are conducted in the presence or absence of anti-TREM2 antibodies or isotype control antibodies.

TREM2-Dependent Gene Expression

In some embodiments, anti-TREM2 antibodies of the present disclosure may increase the activity and/or expression of TREM2-dependent genes, such as one or more transcription factors of the nuclear factor of activated T-cells (NFAT) family of transcription factors.

In some embodiments, the ability of soluble full-length anti-TREM2 antibodies to activate mouse or human TREM2-dependent genes is evaluated using a luciferase reporter gene under the control of an NFAT (nuclear factor of activated T-cells) promoter. In some embodiments, the cell line BW5147.G.1.4 (ATCC® TIB48™), derived from mouse thymus lymphoma T lymphocytes, is infected with mouse TREM2 and DAP12, and with Cignal Lenti NFAT-Luciferase virus (Qiagen). In some embodiments, alternatively the BW5147.G.1.4 cell line is infected with a human TREM2/DAP12 fusion protein, and with Cignal Lenti NFAT-Luciferase virus (Qiagen). In some embodiments, as a positive control for signaling, PMA (0.05 ug/ml) and ionomycin (0.25 uM) are added together. In some embodiments, cells are incubated together with soluble anti-TREM2 and isotype control antibodies for 6 hours and luciferase activity is measured by adding OneGlo Reagent (Promega) to each well and incubating 3 min at room temperature on a plate shaker. In some embodiments, luciferase signal is measured using a BioTek plate reader. In some embodiments, the cells display tonic TREM2-dependent signaling due to either the presence of an endogenous ligand or to spontaneous receptor aggregation, which leads to TREM2 signaling.

In some embodiments, the enhancement of the one or more TREM2 activities induced by binding of one or more TREM2 ligands to the TREM2 protein is measured, for example, utilizing an in vitro cell assay. In some embodiments, the increase in one or more TREM2 activities may be measured by any suitable in vitro cell-based assays or suitable in vivo model described herein or known in the art, for example, by utilizing a luciferase-based reporter assay to measure TREM2-dependent gene expression, using Western blot analysis to measure increase in TREM2-induced phosphorylation of downstream signaling partners, such as Syk, or by utilizing flow cytometry, such as fluorescence-activated cell sorting (FACS) to measure changes in cell surface levels of markers of TREM2 activation. Any in vitro cell-based assays or suitable in vivo model described herein or known in the art may be used to measure interaction (e.g., binding) between TREM2 and one or more TREM2 ligands.

In some embodiments, the increase in one or more TREM2 activities is measured by an in vitro cell-based assay. In some embodiments, to evaluate the ability of anti-TREM2 antibodies to enhance cell survival in vitro, macrophages deficient in the gamma chain subunit of FcgRI, FcgRIII, and FceRI receptors (Fcgr1KO mice, REF: Takai T, Li M, Sylvestre D, Clynes R, Ravetch J. (1994). Cell, 76:519-529) are cultured in the presence of plate-bound anti-TREM2 antibodies and cell viability is determined when cells are cultured in suboptimal growth conditions. In some embodiments, murine bone marrow precursor cells from FcgR1 KO mice (Taconic, Model 584) are obtained by flushing tibial and femoral marrow cells with cold PBS. In some embodiments, after one wash with PBS, erythrocytes are lysed using ACK Lysing Buffer (Lonza), washed twice with PBS and suspended at 0.5×10⁶ cells/ml in complete RPMI media (10% FCS, Pen/Strep, Gln, neAA) with the indicated amount of M-CSF (Peprotech) to produce macrophages. In some embodiments, to analyze cell viability of bone marrow-derived macrophages, cells are prepared as above and plated at 2.5×10⁴/200 μl in a 96-well plate with suboptimal amounts of M-CSF (10 ng/ml) in non-tissue culture treated plates for two days. In some embodiments, cells are then quantified using the ToxGlo™ kit (Promega) and luminescence is determined as a measure of cell viability. In some embodiments, all experiments are conducted in the presence or absence of anti-TREM2 antibodies or isotype control antibodies.

In some embodiments, the increase in one or more TREM2 activities is measured by an in vivo cell-based assay. In some embodiments, to evaluate the ability of anti-TREM2 antibodies to increase the number of immune cells in vivo, C57B16 mice are injected intraperitoneally (IP) with an anti-TREM2 antibody or a mouse IgG1 isotype control antibody, and the number of immune cells in the brain is quantified by FACS. In some embodiments, three to four mice per group receive an IP injection of 40 mg/kg anti-TREM2 antibody or isotype control antibody mIgG1 (clone MOPC-21, Bioxcell). In some embodiments, 48 hours later, the entire brains are harvested, rinsed with PBS, incubated at 37° C. in PBS containing 1 mg/ml collagenase and processed through a cell strainer to obtain a single cell suspension. In some embodiments, cells are then incubated with anti-CD45-PerCp-Cy7, anti-CD11b-PerCP-Cy5.5, anti-Gr1-FITC antibodies and a cell viability dye (Life Technologies, Cat #L34957) for 30 min on ice, then washed twice with cold FACS buffer. In some embodiments, 4% PFA-fixed samples are then analyzed by FACS. In some embodiments, data are acquired on a BD FACSCanto™ II cytometer (Becton Dickinson) and analyzed with FlowJo software.

In some embodiments an anti-TREM2 antibody of the present disclosure enhances one or more TREM2 activities induced by binding of a TREM2 ligand to the TREM2 protein if it induces an increase that ranges from about 1.5-fold to about 6-fold, or more than 6-fold in ligand-induced TREM2-dependent gene transcription when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration. In some embodiments the increase in ligand-induced TREM2-dependent gene transcription is at least 1.5-fold, at least 2-fold, at least a 3-fold, at least a 4-fold, at least a 5-fold, at least a 6-fold, at least a 7-fold, at least a 8-fold, at least a 9-fold, at least a 10-fold, at least an 11-fold, at least a 12-fold, at least a 13-fold, at least a 14-fold, at least a 15-fold, at least a 16-fold, at least a 17-fold, at least an 18-fold, at least a 19-fold, at least a 20-fold or greater when used at a concentration that ranges from about 0.5 nM to about 50 nM, or greater than 50 nM, and as compared to the level of TREM2-dependent gene transcription induced by binding of the TREM2 ligand to the TREM2 protein in the absence of the anti-TREM2 antibody when the TREM2 ligand is used at its EC₅₀ concentration.

In some embodiments, the anti-TREM2 antibody is used at a concentration of at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 15 nM, at least 20 nM, at least 25 nM, at least 30 nM, at least 35 nM, at least 40 nM, at least 45 nM, at least 46 nM, at least 47 nM, at least 48 nM, at least 49 nM, or at least 50 nM. In some embodiments, the TREM2 ligand is phosphatidylserine (PS). In some embodiments, the TREM2 ligand is sphingomyelin (SM). In some embodiments, the increase in one more TEM2 activities may be measured by any suitable in vitro cell-based assays or suitable in vivo model described herein or known in the art. In some embodiments, a luciferase-based reporter assay is used to measure the fold increase of ligand-induced TREM2-dependent gene expression in the presence and absence of antibody, as described in, for example, WO2017/062672 and WO2019/028292.

As used herein, an anti-TREM2 antibody of the present disclosure does not compete with, inhibit, or otherwise block the interaction (e.g., binding) between one or more TREM2 ligands and TREM2 if it decreases ligand binding to TREM2 by less than 20% at saturating antibody concentrations utilizing any in vitro assay or cell-based culture assay described herein or known in the art. In some embodiments, anti-TREM2 antibodies of the present disclosure inhibit interaction (e.g., binding) between one or more TREM2 ligands and TREM2 by less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% at saturating antibody concentrations utilizing any in vitro assay or cell-based culture assay described herein or known in the art.

Anti-TREM2 Antibodies that Decrease Soluble TREM2

In some embodiments, an agonist anti-TREM2 antibody decreases soluble TREM2 (sTREM2). In some embodiments, an agonist anti-TREM2 antibody decreases the level of sTREM2 that is “shed” from the cell surface of a cell into an extracellular sample (e.g. shedding). In some embodiments, such an antibody binds to a region of TREM2 such that it blocks cleavage of TREM2. In such embodiments, the antibody binds to a region comprising His157, the cleavage site of TREM2.

The degree of inhibition of cleavage of TREM2 by an anti-TREM2 antibody negatively correlates with the amount of soluble TREM2 (sTREM2) in the presence of the anti-TREM2 antibody as compared to the amount of sTREM2 in the absence of the anti-TREM2 antibody. For example, an anti-TREM2 antibody may be considered as an anti-TREM2 antibody that inhibits cleavage of TREM2, if in the presence of said anti-TREM2 antibody the amount of sTREM2 is 0-90%, preferably 0-80%, more preferably 0-70%, even more preferably 0-60%, even more preferably 0-50% and even more preferable 0-20% of the amount of sTREM2 in the absence of the anti-TREM2 antibody, as assayed, e.g., by ELISA-based quantification of sTREM2.

In some embodiments, an anti-TREM2 antibody decreases levels of sTREM2 if the amount of sTREM2 in a treated sample is decreased by at least 10%, at least 20%, at least 30%, at least 40%, at least 50%, at least 60%, at least 70%, at least 80%, at least 90% or more as compared to a control value. In some embodiments, an anti-TREM2 antibody decreases levels of sTREM2 if the amount of sTREM2 in a treated sample is decreased by at least 2-fold, 3-fold, 4-fold, 5-fold, 6-fold, 7-fold, 8-fold, 9-fold, 10-fold or more as compared to a control value. In some embodiments, the control value is the amount of sTREM2 in an untreated sample (e.g., a supernatant from a TREM2-expressing cell that has not been treated with an anti-TREM2 antibody, or a sample from a subject that has not been treated with an anti-TREM2 antibody) or a sample treated with an appropriate non-TREM2-binding antibody.

In some embodiments, sTREM2 shedding is measured using a sample that comprises a fluid, e.g., blood, plasma, serum, urine, or cerebrospinal fluid. In some embodiments, the sample comprises cerebrospinal fluid. In some embodiments, the sample comprises supernatant from cell cultures (e.g., supernatant from a primary cell or cell line that endogenously expresses TREM2, such as human macrophages, or a primary cell or cell line that has been engineered to express TREM2.

In some embodiments, the level of sTREM2 in a sample is measured using an immunoassay. Immunoassays are known in the art and include, but are not limited to, enzyme immunoassays (EIA) such as enzyme multiplied immunoassay (EMIA), enzyme linked immunosorbent assay (ELISA), microparticle enzyme immunoassay (MEIA), immunohistochemistry (IHC), immunocytochemistry, capillary electrophoresis immunoassays (CEIA), radioimmunoassays (RIA), immunofluorescence, chemiluminescence immunoassays (CL), and electrochemiluminescence immunoassays (ECL). In some embodiments, sTREM2 levels are measuring using an ELISA assay.

In some embodiments, an ELISA assay can be used for quantitation of levels of sTREM2 in cell culture supernatants. In some embodiments, an ELISA for human sTREM2 is conducted using the Meso Scale Discovery SECTOR Imager 2400. In some embodiments, Streptavidin-coated 96-well plates are blocked overnight at 4° C. in 0.5% bovine serum albumin (BSA) and 0.05% Tween 20 in PBS (pH 7.4) (blocking buffer). In some embodiments, plates are shaken for 1 hour at room temperature with biotinylated polyclonal goat anti-human TREM2 capture antibody (0.25 mg/ml; R&D Systems) diluted in blocking buffer. In some embodiments, plates are washed subsequently four times with 0.05% Tween 20 in PBS (washing buffer) and incubated for 2 hours at room temperature with samples diluted 1:4 in 0.25% BSA and 0.05% Tween 20 in PBS (pH 7.4) (assay buffer) supplemented with protease inhibitors (Sigma). In some embodiments, recombinant human TREM2 protein (Holzel Diagnostika) is diluted in assay buffer in a two-fold serial dilution and used for the standard curve (concentration range, 4000 to 62.5 pg/ml). In some embodiments, plates are washed three times for 5 min with washing buffer before incubation for 1 hour at room temperature with mouse monoclonal anti-TREM2 antibody (1 mg/ml; Santa Cruz Biotechnology; B-3) diluted in blocking buffer. In some embodiments, after three additional washing steps, plates are incubated with a SULFO-TAG-labeled anti-mouse secondary antibody (1:1000; Meso Scale Discovery) and incubated for 1 hour in the dark. In some embodiments, plates are washed three times with washing buffer followed by two washing steps in PBS and developed by adding Meso Scale Discovery Read buffer. In some embodiments, the light emission at 620 nm after electrochemical stimulation is measured using the Meso Scale Discovery SECTOR Imager 2400 reader. In some embodiments, to quantify the levels of sTREM2 secreted, conditioned media from biological replicates are analyzed in duplicates. In some embodiments, sTREM2 standard curves are generated using the MasterPlex ReaderFit software (MiraiBio Group, Hitachi Solutions America) through a five-parameter logistic fit. In some embodiments, levels of sTREM2 are subsequently normalized to levels of immature TREM2 as quantified from Western Blots.

In some embodiments, sTREM2 may be inactive variants of cellular TREM2 receptors. In some embodiments, sTREM2 may be present in the periphery, such as in the plasma, or brains of subject, and may sequester anti-TREM2 antibodies. Such sequestered antibodies would be unable to bind to and activate, for example, the cellular TREM2 receptor present on cells. Accordingly, in certain embodiments, anti-TREM2 antibodies of the present disclosure, such as agonist anti-TREM2 antibodies of the present disclosure, do not bind to soluble TREM2. In some embodiments, anti-TREM2 antibodies of the present disclosure, such as agonist anti-TREM2 antibodies of the present disclosure, do not bind to soluble TREM2 in vivo. In some embodiments, agonist anti-TREM2 antibodies of the present disclosure that do not bind soluble TREM2 may bind to an epitope on TREM2 that, for example, may include a portion of the extracellular domain of cellular TREM2 that is not contained in sTREM2, for example one or more amino acid residues within amino acid residues 161-175; may be at or near a transmembrane portion of TREM2; or may include a transmembrane portion of TREM2.

Antibodies that Affect TREM2 Clustering

In vivo, anti-TREM2 antibodies of the present disclosure may activate receptors by multiple potential mechanisms. In some embodiments, agonistic anti-TREM2 antibodies of the present disclosure, have, due to appropriate epitope specificity, the ability to activate TREM2 in solution without having to be clustered with a secondary antibody, bound on plates, or clustered through Fcg receptors. In some embodiments, anti-TREM2 antibodies of the present disclosure have isotypes of human antibodies, such as IgG2, that have, due to their unique structure, an intrinsic ability to cluster receptors or retain receptors in a clustered configuration, thereby activating receptors such as TREM2 without binding to an Fc receptor (e.g., White et al., (2015) Cancer Cell 27, 138-148).

In certain embodiments, agonist anti-TREM2 antibodies may induce or maintain clustering on the cell surface in order to activate TREM2 and transduce a signal. In certain embodiments, agonist anti-TREM2 antibodies with appropriate epitope specificity may induce or maintain clustering of TREM2 on the cell surface and/or activate TREM2. In some embodiments, agonist anti-TREM2 antibodies bind to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1. In some embodiments, agonist anti-TREM2 antibodies bind to one or more amino acid residues selected from the group consisting of D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1. In some embodiments, anti-TREM2 antibodies of the present disclosure cluster receptors (e.g., TREM2) by binding to Fcg receptors on adjacent cells. Binding of the constant IgG Fc part of the antibody to Fcg receptors leads to aggregation of the antibodies, and the antibodies in turn aggregate the receptors to which they bind through their variable region (Chu et al (2008) Mol Immunol, 45:3926-3933; and Wilson et al., (2011) Cancer Cell 19, 101-113). Any suitable assay known to one of skill in the art (such as those described in WO2017/062672 and WO2019/028292) may be used to determine antibody clustering.

Other mechanisms may also be used to cluster receptors (e.g., TREM2). For example, in some embodiments, antibody fragments (e.g., Fab fragments) that are cross-linked together may be used to cluster receptors (e.g., TREM2) in a manner similar to antibodies with Fc regions that bind Fcg receptors, as described above. In some embodiments, cross-linked antibody fragments (e.g., Fab fragments) may function as agonist antibodies if they induce receptor clustering on the cell surface and bind an appropriate epitope on the target (e.g., TREM2).

An antibody dependent on binding to FcgR receptor to activate targeted receptors may lose its agonist activity if engineered to eliminate FcgR binding (see, e.g., Wilson et al., (2011) Cancer Cell 19, 101-113; Armour at al., (2003) Immunology 40 (2003) 585-593); and White et al., (2015) Cancer Cell 27, 138-148). As such, it is thought that an anti-TREM2 antibody of the present disclosure with appropriate epitope specificity can activate TREM2 when the antibody has an Fc domain.

Exemplary antibody Fc isotypes and modifications are provided in Table A below. In some embodiments, the antibody has an Fc isotype listed in Table A below.

TABLE A
Exemplary antibody Fc isotypes that are
capable of binding Fc gamma receptor

| Fc Isotype | Mutation (EU numbering scheme) |
|---|---|
| IgG1 | N297A |
| IgG1 | D265A and N297A |
| IgG1 | D270A |
| IgG1 | L234A and L235A |
| | L234A and G237A |
| | L234A and L235A and G237A |
| IgG1 | P238D and/or L328E and/or S267E/L328F and/or E233 |
| | and or/G237D and/or H268D and/or P271G and/or A330R |
| IgG1 | P238D and L328E and E233D and G237D and H268D and |
| | P271G and A330R |
| IgG1 | P238D and L328E and G237D and H268D and P271G and |
| | A330R |
| IgG1 | P238D and S267E and L328F and E233D and G237D and |
| | H268D and P271G and A330R |
| IgG1 | P238D and S267E and L328F and G237D and H268D and |
| | P271G and A330R |
| IgG2 | V234A and G237A |
| IgG4 | L235A and G237A and E318A |
| IgG4 | S228P and L236E |
| IgG2/4 | IgG2 aa 118 to 260 and IgG4 aa 261 to 447 |
| hybrid | |
| | H268Q and V309L; and A330S and P331S |
| IgG1 | C226S and C229S and E233P and L234V and L235A |
| IgG1 | L234F and L235E and P331S |
| IgG2 | C232S or C233S |
| IgG2 | A330S and P331S |
| IgG1 | S267E, and L328F |
| | S267E alone |
| IgG2 | S267E and L328F |
| IgG4 | S267E and L328F |
| IgG2 | WT HC with Kappa (light chain) LC |
| | HC C127S with Kappa LC |
| | Kappa LC C214S |
| | Kappa LC C214S and HC C233S |
| | Kappa LC C214S and HC C232S |
| | Any of the above listed mutations together with |
| | A330S and P331S mutations |
| | F(ab′)2 fragment of WT IgG1 and any of the above |
| | listed mutations |
| IgG1 | Substitute the Constant Heavy 1 (CH1) and hinge |
| | region of IgG1 With CH1 and hinge region of IGg2 |
| | ASTKGPSVFP LAPCSRSTSE STAALGCLVK |
| | DYFPEPVTVS WNSGALTSGV HTFPAVLQSS |
| | GLYSLSSVVT VPSSNFGTQT YTCNVDHKPS |
| | NTKVDKTVER KCCVECPPCP (SEQ ID NO: 42) |
| | With a Kappa LC |
| IgG1 | Any of the above listed mutations together with |
| | A330L/A330S and/or L234F and/or L235E and/or P331S |
| IgG1, IgG2, | Any of the above listed mutations together with |
| or IgG4 | M252Y and/or S254T and/or T256E |
| Mouse IgG1 | For mouse disease models |
| IgG4 | WT |
| IgG1 | Any of the above listed mutation together with E430G, |
| | E430S, E430F, E430T, E345K, E345Q, E345R, E345Y, |
| | S440Y, S440W and/or any combination thereof. |
| IgG2 | Any of the above listed mutation together with E430G, |
| | E430S, E430F, E430T, E345K, E345Q, E345R, E345Y, |
| | S440Y, S440W and/or any combination thereof. |

In some embodiments, the antibody is of the IgG class, the IgM class, or the IgA class. In some embodiments, the antibody has an IgG1, IgG2, IgG3, or IgG4 isotype.

Antibodies with human IgG1 or IgG3 isotypes and mutants thereof (e.g. Strohl (2009) Current Opinion in Biotechnology 2009, 20:685-691) that bind the activating Fcg Receptors I, IIA, IIC, IIIA, IIIB in human and/or Fcg Receptors I, III and IV in mouse, may also act as agonist antibodies in vivo but may be associated with effects related to ADCC. However, such Fcg receptors appear to be less available for antibody binding in vivo, as compared to the inhibitory Fcg receptor FcgRIIB (see, e.g., White, et al., (2013) Cancer Immunol. Immunother. 62, 941-948; and Li et al., (2011) Science 333(6045):1030-1034.).

In certain embodiments, the antibody has an IgG2 isotype. In some embodiments, the antibody contains a human IgG2 constant region. In some embodiments, the human IgG2 constant region includes an Fc region. In some embodiments, the antibody induces the one or more TREM2 activities, the DAP12 activities, or both independently of binding to an Fc receptor. In some embodiments, the antibody binds an inhibitory Fc receptor. In certain embodiments, the inhibitory Fc receptor is inhibitory Fc-gamma receptor IIB (FcγIIB), which minimizes or eliminates ADCC. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A (Alegre et al., (1994) Transplantation 57:1537-1543. 31; Xu et al., (2000) Cell Immunol, 200:16-26), G237A (Cole et al. (1999) Transplantation, 68:563-571), H268Q, V309L, A330S, P331S (US 2007/0148167; Armour et al. (1999) Eur J Immunol 29: 2613-2624; Armour et al. (2000) The Haematology Journal 1(Suppl.1):27; Armour et al. (2000) The Haematology Journal 1(Suppl.1):27), C232S, and/or C233S (White et al., (2015) Cancer Cell 27, 138-148), S267E, L328F (Chu et al., (2008) Mol Immunol, 45:3926-3933), M252Y, S254T, and/or T256E, where the amino acid position is according to the EU numbering convention.

In some embodiments, the antibody has an IgG2 isotype with a heavy chain constant domain that contains a C127S amino acid substitution, where the amino acid position is according to the EU numbering convention (White et al., (2015) Cancer Cell 27, 138-148; Lightle et al., (2010) PROTEIN SCIENCE 19:753-762; and WO2008079246).

In some embodiments, the antibody has an IgG2 isotype with a Kappa light chain constant domain that contains a C214S amino acid substitution, where the amino acid position is according to the EU numbering convention (White et al., (2015) Cancer Cell 27, 138-148; Lightle et al., (2010) PROTEIN SCIENCE 19:753-762; and WO2008079246).

In certain embodiments, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor JIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, where the amino acid position is according to the EU numbering convention.

In some embodiments, the antibody includes an IgG2 isotype heavy chain constant domain 1 (CH1) and hinge region (White et al., (2015) Cancer Cell 27, 138-148). In certain embodiments, the IgG2 isotype CH1 and hinge region contain the amino acid sequence of ASTKGPSVFPLAPCSRSTSESTAALGCLVKDYFPEPVTVSWNSGALTSGVHTFPAVLQSSGLY SLSSVVTVPSSNFGTQTYTCNVDHKPSNTKVDKTVERKCCVECPPCP (SEQ ID NO: 42). In some embodiments, the antibody Fc region contains a S267E amino acid substitution, a L328F amino acid substitution, or both, and/or a N297A or N297Q amino acid substitution, where the amino acid position is according to the EU numbering convention.

In certain embodiments, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from L235A, G237A, S228P, L236E (Reddy et al., (2000) J Immunol, 164:1925-1933), S267E, E318A, L328F, M252Y, S254T, and/or T256E, where the amino acid position is according to the EU numbering convention.

In certain embodiments, the antibody has a hybrid IgG2/4 isotype. In some embodiments, the antibody includes an amino acid sequence containing amino acids 118 to 260 according to EU numbering of human IgG2 and amino acids 261-447 according to EU numbering of human IgG4 (WO 1997/11971; WO 2007/106585).

In certain embodiments, the antibody contains a mouse IgG4 constant region (Bartholomaeus, et al. (2014). J. Immunol. 192, 2091-2098).

In some embodiments, the Fc region further contains one or more additional amino acid substitutions selected from A330L, L234F; L235E, or P331S according to EU numbering; and any combination thereof.

In certain embodiments, the antibody contains one or more amino acid substitutions in the Fc region at a residue position selected from C127S, L234A, L234F, L235A, L235E, S267E, K322A, L328F, A330S, P331S, E345R, E430G, S440Y, and any combination thereof, where the numbering of the residues is according to EU numbering. In some embodiments, the Fc region contains an amino acid substitution at positions E430G, L243A, L235A, and P331S, where the numbering of the residue position is according to EU numbering. In some embodiments, the Fc region contains an amino acid substitution at positions E430G and P331S, where the numbering of the residue position is according to EU numbering. In some embodiments, the Fc region contains an amino acid substitution at positions E430G and K322A, where the numbering of the residue position is according to EU numbering. In some embodiments, the Fc region contains an amino acid substitution at positions E430G, A330S, and P331S, where the numbering of the residue position is according to EU numbering. In some embodiments, the Fc region contains an amino acid substitution at positions E430G, K322A, A330S, and P331S, where the numbering of the residue position is according to EU numbering. In some embodiments, the Fc region contains an amino acid substitution at positions E430G, K322A, and A330S, where the numbering of the residue position is according to EU numbering. In some embodiments, the Fc region contains an amino acid substitution at positions E430G, K322A, and P331S, where the numbering of the residue position is according to EU numbering. In some embodiments, the Fc region contains an amino acid substitution at positions S267E and L328F, where the numbering of the residue position is according to EU numbering. In some embodiments, the Fc region contains an amino acid substitution at position C127S, where the numbering of the residue position is according to EU numbering. In some embodiments, the Fc region contains an amino acid substitution at positions E345R, E430G and S440Y, where the numbering of the residue position is according to EU numbering.

In some embodiments, the antibody has a human IgG1 isotype and comprises amino acid substitutions in the Fc region at the residue positions P331S and E430G, wherein the numbering of the residues is according to EU numbering. An Fc region comprising amino acid substitutions at the residue positions P331S and E430G may be referred to as “PSEG.”

Further IgG Mutations

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU numbering convention, to eliminate complement activation. In some embodiments, the IgG variants described herein may be combined with one or more mutations to enhance the antibody half-life in human serum (e.g. M252Y, S254T, T256E mutations according to the EU numbering convention) (Dall'Acqua et al., (2006) J Biol Chem, 281:23514-23524; and Strohl e al., (2009) Current Opinion in Biotechnology, 20:685-691).

In some embodiments, an IgG4 variant of the present disclosure may be combined with an S228P mutation according to the EU numbering convention (Angal et al., (1993) Mol Immunol, 30:105-108) and/or with one or more mutations described in Peters et al., (2012) J Biol Chem. 13; 287(29):24525-33) to enhance antibody stabilization.

Exemplary Anti-TREM2 Antibodies

In some embodiments, an anti-TREM2 antibody of the present disclosure binds to TREM2 with high affinity, is an agonist, and induces one or more TREM2 activities. In some embodiments, the anti-TREM2 antibody enhances one or more TREM2 activities induced by binding of one or more TREM2 ligands to the TREM2 protein, as compared to the one or more TREM2 activities induced by binding of the one or more TREM2 ligands to the TREM2 protein in the absence of the isolated antibody. In some embodiments, the anti-TREM2 antibody enhances the one or more TREM2 activities without competing with or otherwise blocking binding of the one or more TREM2 ligands to the TREM2 protein. In some embodiments, the antibody is a humanized antibody, a bispecific antibody, a multivalent antibody, or a chimeric antibody. Exemplary descriptions of such antibodies are found throughout the present disclosure. In some embodiments, the antibody is a bispecific antibody recognizing a first antigen and a second antigen.

In some embodiments, anti-TREM2 antibodies of the present disclosure bind to a human TREM2, or a homolog thereof, including without limitation a mammalian (e.g., non-human mammalian) TREM2 protein, mouse TREM2 protein (Uniprot Accession No. Q99NH8), rat TREM2 protein (Uniprot Accession No. D3ZZ89), Rhesus monkey TREM2 protein (Uniprot Accession No. F6QVF2), cynomolgus monkey TREM2 protein (NCBI Accession No. XP_015304909.1), equine TREM2 protein (Uniprot Accession No. F7D6L0), pig TREM2 protein (Uniprot Accession No. H2EZZ3), and dog TREM2 protein (Uniprot Accession No. E2RP46). In some embodiments, anti-TREM2 antibodies of the present disclosure specifically bind to human TREM2. In some embodiments, anti-TREM2 antibodies of the present disclosure specifically bind to cynomolgus monkey TREM2. In some embodiments, anti-TREM2 antibodies of the present disclosure specifically bind to both human TREM2 and cynomolgus monkey TREM2. In some embodiments, anti-TREM2 antibodies of the present disclosure induce at least one TREM2 activity of the present disclosure.

Anti-TREM2 Antibody-Binding Regions

In some embodiments, anti-TREM2 antibodies of the present disclosure bind to one or more amino acids within amino acid residues 124-153 of SEQ ID NO: 1, or amino acid residues on a TREM2 protein corresponding to amino acid residues 124-153 of SEQ ID NO: 1; one or more amino acids within amino acid residues 129-153 of SEQ ID NO: 1, or amino acid residues on a TREM2 protein corresponding to amino acid residues 129-153 of SEQ ID NO: 1; one or more amino acids within amino acid residues 140-149 of SEQ ID NO: 1, or amino acid residues on a TREM2 protein corresponding to amino acid residues 140-149 of SEQ ID NO: 1; one or more amino acids within amino acid residues 149-157 of SEQ ID NO: 1, or amino acid residues on a TREM2 protein corresponding to amino acid residues 149-157 of SEQ ID NO: 1; or one or more amino acids within amino acid residues 153-162 of SEQ ID NO: 1, or amino acid residues on a TREM2 protein corresponding to amino acid residues 153-162 of SEQ ID NO: 1. In some embodiments, anti-TREM2 antibodies of the present disclosure bind one or more of amino acid residues D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1.

Anti-TREM2 Antibody Light Chain and Heavy Chain Variable Regions

In some embodiments, anti-TREM2 antibodies to be used in the methods of the present disclosure are described in WO2019/028292, which is hereby incorporated by reference herein. In some embodiments, the anti-TREM2 antibodies to be used in the methods of the present disclosure induce or enhance one or more of the following TREM2 activities: TREM2 binding to DAP12; DAP12 phosphorylation; activation of Syk kinase; modulation of one or more pro-inflammatory mediators selected from IFN-β, IL-1α, IL-1β, TNF-α, YM-1, IL-6, IL-8, CRP, CD86, MCP-1/CCL2, CCL3, CCL4, CCL5, CCR2, CXCL-10, Gata3, Rorc, IL-20 family members, IL-33, LIF, IFN-gamma, OSM, CNTF, GM-CSF, CSF-1, MHC-II, OPN, CD11c, GM-CSF, IL-11, IL-12, IL-17, IL-18, and IL-23, optionally where the modulation occurs in one or more cells selected from macrophages, M1 macrophages, activated M1 macrophages, M2 macrophages, dendritic cells, monocytes, osteoclasts, Langerhans cells of skin, Kupffer cells, and microglial cells; recruitment of Syk, ZAP70, or both to a DAP12/TREM2 complex; increasing activity of one or more TREM2-dependent genes, optionally where the one or more TREM2-dependent genes comprise nuclear factor of activated T-cells (NFAT) transcription factors; increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof; modulated expression of one or more stimulatory molecules selected from CD83, CD86 MHC class II, CD40, and any combination thereof, optionally where the CD40 is expressed on dendritic cells, monocytes, macrophages, or any combination thereof, and optionally where the dendritic cells comprise bone marrow-derived dendritic cells; increasing memory; and reducing cognitive deficit. In some embodiments, anti-TREM2 antibodies of the present disclosure increase memory and/or reduce cognitive deficit when administered to an individual.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 34; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 35; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 31; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 41; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 33; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 36; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 37; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 38; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 39; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 40; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 32.

In some embodiments, anti-TREM2 antibodies of the present disclosure comprise a light chain variable domain and a heavy chain variable domain, wherein the heavy chain variable region comprises an HVR-H1 comprising the amino acid sequence YAFSSDWMN (SEQ ID NO: 36), an HVR-H2 comprising the amino acid sequence RIYPGEGDTNYARKFHG (SEQ ID NO: 37), an HVR-H3 comprising the amino acid sequence ARLLRNKPGESYAMDY (SEQ ID NO: 38), and the light chain variable region comprises an HVR-L1 comprising the amino acid sequence RTSQSLVHSNAYTYLH (SEQ ID NO: 39), an HVR-L2 comprising the amino acid sequence KVSNRVS (SEQ ID NO: 40), and an HVR-L3 comprising the amino acid sequence SQSTRVPYT (SEQ ID NO: 32). In some embodiments, anti-TREM2 antibodies of the present disclosure comprise a light chain variable domain and a heavy chain variable domain, wherein the heavy chain variable region comprises an HVR-H1 comprising the amino acid sequence YAFSSQWMN (SEQ ID NO: 34), an HVR-H2 comprising the amino acid sequence RIYPGGGDTNYAGKFQG (SEQ ID NO: 35), an HVR-H3 comprising the amino acid sequence ARLLRNQPGESYAMDY (SEQ ID NO: 31), and the light chain variable region comprises an HVR-L1 comprising the amino acid sequence RSSQSLVHSNRYTYLH (SEQ ID NO: 41), an HVR-L2 comprising the amino acid sequence KVSNRFS (SEQ ID NO: 33), and an HVR-L3 comprising the amino acid sequence SQSTRVPYT (SEQ ID NO: 32). In some embodiments, anti-TREM2 antibodies of the present disclosure comprise a heavy chain variable region and a light chain variable region, wherein the heavy chain variable region comprises one, two, three or four frame work regions selected from VH FR1, VH FR2, VH FR3, and VH FR4, wherein: the VH FR1 comprises a sequence selected from the group consisting of SEQ ID NOs: 9-11, the VH FR2 comprises a sequence selected from the group consisting of SEQ ID NOs: 12 and 13, the VH FR3 comprises a sequence selected from the group consisting of SEQ ID NOs: 14 and 15, and the VH FR4 comprises the sequence of SEQ ID NO: 16; and/or the light chain variable region comprises one, two, three or four frame work regions selected from VL FR1, VL FR2, VL FR3, and VL FR4, wherein: the L FR1 comprises a sequence selected from the group consisting of SEQ ID NOs: 17-20, the VL FR2 comprises a sequence selected from the group consisting of SEQ ID NOs: 21 and 22, the VL FR3 comprises a sequence selected from the group consisting of SEQ ID NOs: 23 and 24, and the VL FR4 comprises a sequence selected from the group consisting of SEQ ID NOs: 25 and 26.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28, wherein the heavy chain variable domain comprises the HVR-H1, HVR-H2, and HVR-H3 amino acid sequences of antibody AL2p-47. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29, wherein the light chain variable domain comprises the HVR-L1, HVR-L2, and HVR-L3 amino acid sequences of antibody AL2p-47. In some embodiments, the anti-TREM2 antibody comprises a heavy chain variable domain (V_{H}) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-47 or to the amino acid sequence of SEQ ID NO: 28 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AL2p-47 or the amino acid sequence of SEQ ID NO: 28. In certain embodiments, a total of 1 to 5 amino acids have been substituted, inserted and/or deleted in the heavy chain variable domain amino acid sequence of antibody AL2p-47 or the amino acid sequence of SEQ ID NO: 28. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AL2p-47 or of SEQ ID NO: 28, including post-translational modifications of that sequence. In a particular embodiment, the VH comprises one, two or three HVRs selected from: (a) the HVR-H1 amino acid sequence of antibody AL2p-47, (b) the HVR-H2 amino acid sequence of antibody AL2p-47, and (c) the HVR-H3 amino acid sequence of antibody AL2p-47. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-47 or to the amino acid sequence of SEQ ID NO: 29 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AL2p-47 or the amino acid sequence of SEQ ID NO: 29. In certain embodiments, a total of 1 to 5 amino acids have been substituted, inserted and/or deleted in the light chain variable domain amino acid sequence of antibody AL2p-47 or the amino acid sequence of SEQ ID NO: 29. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AL2p-47 or of SEQ ID NO: 29, including post-translational modifications of that sequence. In a particular embodiment, the VL comprises one, two or three HVRs selected from: (a) the HVR-L1 amino acid sequence of antibody AL2p-47, (b) the HVR-L2 amino acid sequence of antibody AL2p-47, and (c) the HVR-L3 amino acid sequence of antibody AL2p-47. In some embodiments, the antibody comprises a heavy chain variable region comprising the amino acid sequence of SEQ ID NO: 28 and a light chain variable region comprising the amino acid sequence of SEQ ID NO: 29.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27, wherein the heavy chain variable domain comprises the HVR-H1, HVR-H2, and HVR-H3 amino acid sequences of antibody AL2p-58. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30, wherein the light chain variable domain comprises the HVR-L1, HVR-L2, and HVR-L3 amino acid sequences of antibody AL2p-58. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody AL2p-58 or to the amino acid sequence of SEQ ID NO: 27 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody AL2p-58 or the amino acid sequence of SEQ ID NO: 27. In certain embodiments, a total of 1 to 5 amino acids have been substituted, inserted and/or deleted in the heavy chain variable domain amino acid sequence of antibody AL2p-58 or the amino acid sequence of SEQ ID NO: 27. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody AL2p-58 or of SEQ ID NO: 27, including post-translational modifications of that sequence. In a particular embodiment, the VH comprises one, two or three HVRs selected from: (a) the HVR-H1 amino acid sequence of antibody AL2p-58, (b) the HVR-H2 amino acid sequence of antibody AL2p-58, and (c) the HVR-H3 amino acid sequence of antibody AL2p-58. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody AL2p-58 or to the amino acid sequence of SEQ ID NO: 30 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody AL2p-58 or the amino acid sequence of SEQ ID NO: 30. In certain embodiments, a total of 1 to 5 amino acids have been substituted, inserted and/or deleted in the light chain variable domain amino acid sequence of antibody AL2p-58 or the amino acid sequence of SEQ ID NO: 30. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody AL2p-58 or of SEQ ID NO: 30, including post-translational modifications of that sequence. In a particular embodiment, the VL comprises one, two or three HVRs selected from: (a) the HVR-L1 amino acid sequence of antibody AL2p-58, (b) the HVR-L2 amino acid sequence of antibody AL2p-58, and (c) the HVR-L3 amino acid sequence of antibody AL2p-58. In some embodiments, the antibody comprises a heavy chain variable region comprising the amino acid sequence of SEQ ID NO: 27 and a light chain variable region comprising the amino acid sequence of SEQ ID NO: 30.

In some embodiments, the antibody comprises a heavy chain comprising the amino acid of SEQ ID NO: 43, and a light chain comprising the amino acid sequence of SEQ ID NO: 47; or a heavy chain comprising the amino acid of SEQ ID NO: 44, and a light chain comprising the amino acid sequence of SEQ ID NO: 47.

In some embodiments, the antibody comprises a heavy chain comprising the amino acid of SEQ ID NO: 45, and a light chain comprising the amino acid sequence of SEQ ID NO: 48; or a heavy chain comprising the amino acid of SEQ ID NO: 46, and a light chain comprising the amino acid sequence of SEQ ID NO: 48.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 50; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 51; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 52; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 53; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 54; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 55.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 58; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 59; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 60; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 61; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 62; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 63.

In some embodiments, anti-TREM2 antibodies of the present disclosure comprise a light chain variable domain and a heavy chain variable domain, wherein the heavy chain variable domain comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 66; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 67; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 68; and/or wherein the light chain variable domain comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 69; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 70; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 71.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 56; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 57. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 56, wherein the heavy chain variable domain comprises the HVR-H1, HVR-H2, and HVR-H3 amino acid sequences of antibody 42E8.H1. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 57, wherein the light chain variable domain comprises the HVR-L1, HVR-L2, and HVR-L3 amino acid sequences of antibody 42E8.H1. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody 42E8.H1 or to the amino acid sequence of SEQ ID NO: 56 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody 42E8.H1 or the amino acid sequence of SEQ ID NO: 56. In certain embodiments, a total of 1 to 5 amino acids have been substituted, inserted and/or deleted in the heavy chain variable domain amino acid sequence of antibody 42E8.H1 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody 42E8.H1 or of SEQ ID NO: 56, including post-translational modifications of that sequence. In a particular embodiment, the VH comprises one, two or three HVRs selected from: (a) the HVR-H1 amino acid sequence of antibody 42E8.H1, (b) the HVR-H2 amino acid sequence of antibody 42E8.H1, and (c) the HVR-H3 amino acid sequence of antibody 42E8.H1. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody 42E8.H1 or to the amino acid sequence of SEQ ID NO: 57 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody 42E8.H1 or the amino acid sequence of SEQ ID NO: 57. In certain embodiments, a total of 1 to 5 amino acids have been substituted, inserted and/or deleted in the light chain variable domain amino acid sequence of antibody 42E8.H1 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody 42E8.H1 or of SEQ ID NO: 57, including post-translational modifications of that sequence. In a particular embodiment, the VL comprises one, two or three HVRs selected from: (a) the HVR-L1 amino acid sequence of antibody 42E8.H1, (b) the HVR-L2 amino acid sequence of antibody 42E8.H1, and (c) the HVR-L3 amino acid sequence of antibody 42E8.H1. In some embodiments, the antibody comprises a heavy chain variable region comprising the amino acid sequence of SEQ ID NO: 56 and a light chain variable region comprising the amino acid sequence of SEQ ID NO: 57.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 64; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 65. In some embodiments, anti-TREM2 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 64, wherein the heavy chain variable domain comprises the HVR-H1, HVR-H2, and HVR-H3 amino acid sequences of antibody RS9.F6. In some embodiments, anti-TREM2 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 65, wherein the light chain variable domain comprises the HVR-L1, HVR-L2, and HVR-L3 amino acid sequences of antibody RS9.F6. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a heavy chain variable domain amino acid sequence of antibody RS9.F6 or to the amino acid sequence of SEQ ID NO: 64 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the heavy chain variable domain amino acid sequence of antibody RS9.F6 or the amino acid sequence of SEQ ID NO: 64. In certain embodiments, a total of 1 to 5 amino acids have been substituted, inserted and/or deleted in the heavy chain variable domain amino acid sequence of antibody RS9.F6 or the amino acid sequence of SEQ ID NO: 64. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of antibody RS9.F6 or of SEQ ID NO: 64, including post-translational modifications of that sequence. In a particular embodiment, the VH comprises one, two or three HVRs selected from: (a) the HVR-H1 amino acid sequence of antibody RS9.F6, (b) the HVR-H2 amino acid sequence of antibody RS9.F6, and (c) the HVR-H3 amino acid sequence of antibody RS9.F6. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a light chain variable domain amino acid sequence of antibody RS9.F6 or to the amino acid sequence of SEQ ID NO: 65 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the light chain variable domain amino acid sequence of antibody RS9.F6 or the amino acid sequence of SEQ ID NO: 65. In certain embodiments, a total of 1 to 5 amino acids have been substituted, inserted and/or deleted in the light chain variable domain amino acid sequence of antibody RS9.F6 or the amino acid sequence of SEQ ID NO: 65. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of antibody RS9.F6 or of SEQ ID NO: 65, including post-translational modifications of that sequence. In a particular embodiment, the VL comprises one, two or three HVRs selected from: (a) the HVR-L1 amino acid sequence of antibody RS9.F6, (b) the HVR-L2 amino acid sequence of antibody RS9.F6, and (c) the HVR-L3 amino acid sequence of antibody RS9.F6. In some embodiments, the antibody comprises a heavy chain variable region comprising the amino acid sequence of SEQ ID NO: 64 and a light chain variable region comprising the amino acid sequence of SEQ ID NO: 65.

In some embodiments, anti-TREM2 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 72; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 73. In some embodiments, the anti-TREM2 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 72 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 72. In certain embodiments, a total of 1 to 5 amino acids have been substituted, inserted and/or deleted in the amino acid sequence of SEQ ID NO: 72. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VH sequence of SEQ ID NO: 72, including post-translational modifications of that sequence. In some embodiments, anti-TREM2 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to the amino acid sequence of SEQ ID NO: 73 and contains substitutions (e.g., conservative substitutions, insertions, or deletions relative to the reference sequence), but the anti-TREM2 antibody comprising that sequence retains the ability to bind to TREM2. In certain embodiments, a total of 1 to 10 amino acids have been substituted, inserted, and/or deleted in the amino acid sequence of SEQ ID NO: 73. In certain embodiments, a total of 1 to 5 amino acids have been substituted, inserted and/or deleted in the amino acid sequence of SEQ ID NO: 73. In certain embodiments, substitutions, insertions, or deletions occur in regions outside the HVRs (i.e., in the FR regions). In some embodiments, the substitutions, insertions, or deletions occur in in the FR regions. Optionally, the anti-TREM2 antibody comprises the VL sequence of SEQ ID NO: 73, including post-translational modifications of that sequence. In some embodiments, the antibody comprises a heavy chain variable region comprising the amino acid sequence of SEQ ID NO: 72 and a light chain variable region comprising the amino acid sequence of SEQ ID NO: 73.

In some embodiments, an agonist anti-TREM2 antibody of the present disclosure is AL2p-58 huIgG1 PSEG. In some embodiments, an agonist anti-TREM2 antibody of the present disclosure is AL2p-47 IhuIgG1.

TABLE B
Sequences

| SEQ ID NO | Sequence | Description |
|---|---|---|
| 1 | MEPLRLLILLFVTELSGAHNTTVFQGVAGQSLQVSCPYDS | Human TREM2 |
| | MKHWGRRKAWCRQLGEKGPCQRVVSTHNLWLLSFLRR | protein |
| | WNGSTAITDDTLGGTLTITLRNLQPHDAGLYQCQSLHGSE | |
| | ADTLRKVLVEVLADPLDHRDAGDLWFPGESESFEDAHVE | |
| | HSISRSLLEGEIPFPPTSILLLLACIFLIKILAASALWAAAWH | |
| | GQKPGTHPPSELDCGHDPGYQLQTLPGLRDT | |
| 2 | MGPLHQFLLLLITALSQALNTTVLQGMAGQSLRVSCTYD | Mouse TREM2 |
| | ALKHWGRRKAWCRQLGEEGPCQRVVSTHGVWLLAFLK | protein |
| | KRNGSTVIADDTLAGTVTITLKNLQAGDAGLYQCQSLRG | |
| | REAEVLQKVLVEVLEDPLDDQDAGDLWVPEESSSFEGAQ | |
| | VEHSTSRNQETSFPPTSILLLLACVLLSKFLAASILWAVAR | |
| | GRQKPGTPVVRGLDCGQDAGHQLQILTGPGGT | |
| 3 | MEPLHVFVLLLVTELSQALNTTVLQGVAGQSLRVSCTYD | Rat TREM2 protein |
| | ALRHWGRRKAWCRQLAEEGPCQRVVSTHGVWLLAFLRK | |
| | QNGSTVITDDTLAGTVTITLRNLQAGDAGLYQCQSLRGRE | |
| | AEVLQKVVVEVLEDPLDDQDAGDLWVPEESESFEGAQVE | |
| | HSTSSQVSSCGSPLTYHLPPKEPIRKDLLPTHFHSSPPGLCP | |
| | PEQASYSQHPLGCGQGQAEAGDTCGQWARL | |
| 4 | MPDPLFSAVQGKDKILHKALCICPWPGKGGMEPLRLLILL | Rhesus monkey |
| | FATELSGAHNTTVFQGVEGQSLQVSCPYDSMKHWGRRK | TREM2 protein |
| | AWCRQLGEKGPCQRVVSTHNLWLLSFLRRRNGSTAITDD | |
| | TLGGTLTITLRNLQPHDAGFYQCQSLHGSEADTLRKVLVE | |
| | VLADPLDHRDAGDLWVPGESESFEDAHVEHSISRSLLEGE | |
| | IPFPPTSVLLLLACIFLIKILAASALWAAAWHGQKPGTHPPS | |
| | EPDCGHDPGHQLQTLPGLRDT | |
| 5 | MEPLRLLILLFATELSGAHNTTVFQGVEGQSLQVSCPYDS | Cynomolgus monkey |
| | MKHWGRRKAWCRQLGEKGPCQRVVSTHNLWLLSFLRRR | TREM2 protein |
| | NGSTAITDDTLGGTLTITLRNLQPHDAGFYQCQSLHGSEA | |
| | DTLRKVLVEVLADPLDHRDAGDLWVPGESESFEDAHVEH | |
| | SISRSLLEGEIPFPPTSVLLLLACIFLIKILAASALWAAAWH | |
| | GQKPGTHPPSEPDCGHDPGHQLQTLPGLRDT | |
| 6 | MEPLPLLILLSVAELSRGHNTTVFQGTAGRSLKVSCPYNSL | Equine TREM2 |
| | MHWGRRKAWCRQLGEDGPCQQVVSTHSLWLLSFLKRRN | protein |
| | GSTVITDDALGGILTITLRNLQAHDAGFYQCQSLHGGEAD | |
| | TLRKVLVEVLADPLDHQEPGDLWIPKESESFEDAQVEHSIS | |
| | RSLVEEEIPSLPTSILLLLACIFLSKLLAASAIWAAAWHGQK | |
| | QETPPASEPDRGHDPGYQLHTLTGERDT | |
| 7 | METLGLLLLLWVAELSRAHNTSVFQGTAGQSLRVSCSYN | Pig TREM2 protein |
| | SLKHWGRRKAWCRQLSEEGLCQHVVSTHPTWLLSFLKRR | |
| | NGSTAITDDALGGTLTITLRNLQAHDAGLYQCQSLHGSEA | |
| | DTLKKVLVEVLADPLESQSKSFQDVQMEHSISRNLSEESLF | |
| | PPTSTLFLLACVFLSKLLVASALWAAAWHGHKQRTSPAG | |
| | GLDCGRDPGDQDQTLTDELGESSDQDQTLTELRDT | |
| 8 | MEPLWLLILLAVTELSGAHNTTVFQGMAGRSLQVSCPYN | Dog TREM2 protein |
| | SLKHWGRRKAWCRQVDKEGPCQRVVSTHRSWLLSFLKR | |
| | WNGSTAIVDDALGGTLTITLRNLQAHDAGLYQCQSLYGD | |
| | EADTLRKVLVEVLADPLDHLDPGDLWIPEESKGFEDAHV | |
| | EPSVSRSLSEEEIPFPPTSILFLLACIFLSKFLAASALWAAA | |
| | WRGQKLGTPQASELDCSCDPGYQLQTLTEPRDM | |
| 9 | QVQLVQSGAEVKKPGSSVKVSCKASG | VHFR1 |
| 10 | EVQLVQSGAEVKKPGSSVKVSCKASG | VHFR1 |
| 11 | QVQLVQSGAEVKKPGASVKVSCKASG | VHFR1 |
| 12 | WVRQAPGQGLEWMG | VHFR2 |
| 13 | WVRQAPGQRLEWIG | VHFR2 |
| 14 | RVTITADESTSTAYMELSSLRSEDTAVYYC | VHFR3 |
| 15 | RVTITADTSASTAYMELSSLRSEDTAVYYC | VHFR3 |
| 16 | WGQGTLVTVSS | VHFR4 |
| 17 | DVVMTQTPLSLSVTPGQPASISC | VLFR1 |
| 18 | GVVMTQTPLSLSVTPGQPASISC | VLFR1 |
| 19 | GVVMAQTPLSLSVTPGQPASISC | VLFR1 |
| 20 | DVVMTQSPDSLAVSLGERATINC | VLFR1 |
| 21 | WYLQKPGQSPQLLIY | VLFR2 |
| 22 | WYQQKPGQSPKLLIY | VLFR2 |
| 23 | GVPDRFSGSGSGTDFTLKISRVEAEDVGVYYC | VLFR3 |
| 24 | GVPDRFSGSGSGTDFTLTISSLQAEDVAVYYC | VLFR3 |
| 25 | FGQGTKLEIK | VLFR4 |
| 26 | FGGGTKVEIK | VLFR4 |
| 27 | QVQLVQSGAEVKKPGASVKVSCKASGYAFSSQWMNWV | AL2p-58 - Heavy |
| | RQAPGQRLEWIGRIYPGGGDTNYAGKFQGRVTITADTSAS | chain variable |
| | TAYMELSSLRSEDTAVYYCARLLRNQPGESYAMDYWGQ | domain |
| | GTLVTVSS | |
| 28 | QVQLVQSGAEVKKPGSSVKVSCKASGYAFSSDWMNWVR | AL2p-47 - Heavy |
| | QAPGQGLEWMGRIYPGEGDTNYARKFHGRVTITADESTS | chain variable |
| | TAYMELSSLRSEDTAVYYCARLLRNKPGESYAMDYWGQ | domain |
| | GTLVTVSS | |
| 29 | DVVMTQTPLSLSVTPGQPASISCRTSQSLVHSNAYTYLHW | AL2p-47 - Light |
| | YLQKPGQSPQLLIYKVSNRVSGVPDRFSGSGSGTDFTLKIS | chain variable |
| | RVEAEDVGVYYCSQSTRVPYTFGQGTKLEIK | domain |
| 30 | DVVMTQSPDSLAVSLGERATINCRSSQSLVHSNRYTYLH | AL2p-58 - Light |
| | WYQQKPGQSPKLLIYKVSNRFSGVPDRFSGSGSGTDFTLK | chain variable |
| | ISRVEAEDVGVYYCSQSTRVPYTFGQGTKLEIK | domain |
| 31 | ARLLRNQPGESYAMDY | AL2p-58 - HVR-H3 |
| 32 | SQSTRVPYT | AL2p-58; AL2p-47 - |
| | | HVR-L3 |
| 33 | KVSNRFS | AL2p-58 - HVR-L2 |
| 34 | YAFSSQWMN | AL2p-58 - HVR-H1 |
| 35 | RIYPGGGDTNYAGKFQG | AL2p-58 - HVR-H2 |
| 36 | YAFSSDWMN | AL2p-47 - HVR-H1 |
| 37 | RIYPGEGDTNYARKFHG | AL2p-47 - HVR-H2 |
| 38 | ARLLRNKPGESYAMDY | AL2p-47 - HVR-H3 |
| 39 | RTSQSLVHSNAYTYLH | AL2p-47 - HVR-LI |
| 40 | KVSNRVS | AL2p-47 - HVR-L2 |
| 41 | RSSQSLVHSNRYTYLH | AL2p-58 - HVR-LI |
| 42 | ASTKGPSVFPLAPCSRSTSESTAALGCLVKDYFPEPVTVSW | IgG2 isotype heavy |
| | NSGALTSGVHTFPAVLQSSGLYSLSSVVTVPSSNFGTQTYT | chain constant |
| | CNVDHKPSNTKVDKTVERKCCVECPPCP | domain 1 (CH1) and |
| | | hinge region |
| 43 | QVQLVQSGAEVKKPGASVKVSCKASGYAFSSQWMNWV | AL2p-58 huIgG1 |
| | RQAPGQRLEWIGRIYPGGGDTNYAGKFQGRVTITADTSAS | PSEG - Heavy chain |
| | TAYMELSSLRSEDTAVYYCARLLRNQPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPASIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHGALHNHYTQKSLSLSPGK | |
| 44 | QVQLVQSGAEVKKPGASVKVSCKASGYAFSSQWMNWV | AL2p-58 huIgG1 |
| | RQAPGQRLEWIGRIYPGGGDTNYAGKFQGRVTITADTSAS | PSEG - Heavy chain |
| | TAYMELSSLRSEDTAVYYCARLLRNQPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPASIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHGALHNHYTQKSLSLSPG | |
| 45 | QVQLVQSGAEVKKPGSSVKVSCKASGYAFSSDWMNWVR | AL2p-47 huIgG1 - |
| | QAPGQGLEWMGRIYPGEGDTNYARKFHGRVTITADESTS | Heavy chain |
| | TAYMELSSLRSEDTAVYYCARLLRNKPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPAPIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSPGK | |
| 46 | QVQLVQSGAEVKKPGSSVKVSCKASGYAFSSDWMNWVR | AL2p-47 huIgG1 - |
| | QAPGQGLEWMGRIYPGEGDTNYARKFHGRVTITADESTS | Heavy chain |
| | TAYMELSSLRSEDTAVYYCARLLRNKPGESYAMDYWGQ | |
| | GTLVTVSSASTKGPSVFPLAPSSKSTSGGTAALGCLVKDY | |
| | FPEPVTVSWNSGALTSGVHTFPAVLQSSGLYSLSSVVTVPS | |
| | SSLGTQTYICNVNHKPSNTKVDKKVEPKSCDKTHTCPPCP | |
| | APELLGGPSVFLFPPKPKDTLMISRTPEVTCVVVDVSHEDP | |
| | EVKFNWYVDGVEVHNAKTKPREEQYNSTYRVVSVLTVL | |
| | HQDWLNGKEYKCKVSNKALPAPIEKTISKAKGQPREPQV | |
| | YTLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSPG | |
| 47 | DVVMTQSPDSLAVSLGERATINCRSSQSLVHSNRYTYLH | AL2p-58 huIgG1 |
| | WYQQKPGQSPKLLIYKVSNRFSGVPDRFSGSGSGTDFTLK | PSEG - Light chain |
| | ISRVEAEDVGVYYCSQSTRVPYTFGQGTKLEIKRTVAAPS | |
| | VFIFPPSDEQLKSGTASVVCLLNNFYPREAKVQWKVDNAL | |
| | QSGNSQESVTEQDSKDSTYSLSSTLTLSKADYEKHKVYAC | |
| | EVTHQGLSSPVTKSFNRGEC | |
| 48 | DVVMTQTPLSLSVTPGQPASISCRTSQSLVHSNAYTYLHW | AL2p-47 huIgG1 - |
| | YLQKPGQSPQLLIYKVSNRVSGVPDRFSGSGSGTDFTLKIS | Light chain |
| | RVEAEDVGVYYCSQSTRVPYTFGQGTKLEIKRTVAAPSVF | |
| | IFPPSDEQLKSGTASVVCLLNNFYPREAKVQWKVDNALQS | |
| | GNSQESVTEQDSKDSTYSLSSTLTLSKADYEKHKVYACEV | |
| | THQGLSSPVTKSFNRGEC | |
| 49 | D/Ex0-2YxxL/IX6-8YxxL/I | Receptor motif |
| 50 | GYSITSDYAWN | 42E8.H1 CDR-H1 |
| 51 | YINYSGRTIYNPSLKS | 42E8.H1 CDR-H2 |
| 52 | ARWNGNYGFAY | 42E8.H1 CDR-H3 |
| 53 | RSSQSLVHINGNTYLH | 42E8.H1 CDR-L1 |
| 54 | KVSNRFS | 42E8.H1 CDR-L2 |
| 55 | SQTTHALFT | 42E8.H1 CDR-L3 |
| 56 | DVQLQESGPGLVKPSQSLSLTCTVTGYSITSDYAWNWIRQ | 42E8.H1 |
| | FPGNKLEWMGYINYSGRTIYNPSLKSRISITRDTSKNHFFL | Heavy Chain |
| | QLISVTTEDTATYYCARWNGNYGFAYWGQGTLVTVSA | Variable Region |
| 57 | DWMTQNPLSLPVSLGDQASISCRSSQSLVHINGNTYLHWY | 42E8.H1 |
| | LQKPGQSPKLLIYKVSNRFSGVPDRFSGSGSGTDFTLKISR | Light Chain Variable |
| | VEAEDLGVYFCSQTTHALFTFGSGTKLEIK | Region |
| 58 | GYTFTSY | RS9.F6 CDR-H1 |
| 59 | IGRSDPTTGGTNYNE | RS9.F6 CDR-H2 |
| 60 | VRTSGTGDY | RS9.F6 CDR-H3 |
| 61 | RSSQSLVHNNGNTFLH | RS9.F6 CDR-L1 |
| 62 | VSNRFS | RS9.F6 CDR-L2 |
| 63 | SQTTHVPPT | RS9.F6 CDR-L3 |
| 64 | QVQLQQPGAELVKPGASVKLSCKASGYTFTSYWMHWVK | RS9.F6 |
| | QSPGRGLEWIGRSDPTTGGTNYNEKFKTKATLTVDKPSST | Heavy Chain |
| | AYMQLSSLTSDDSAVYYCVRTSGTGDYWGQGTSLTVSSA | Variable Region |
| | KTTAPSVYPLAPVCGGTTGSSVT | |
| 65 | DVVMTQTPLSLPVSLGDQASISCRSSQSLVHNNGNTFLHW | RS9.F6 |
| | YLQKPGQSPKLLIYKVSNRFSGVPDRFSGSGSGTDFTLKIS | Light Chain Variable |
| | RVEAEDLGVYFCSQTTHVPPTFGGGTKLEIKRADAAPTVS | Region |
| | IFPPSSEQLTSGGASVVCF | |
| 66 | GFTFTDFY | WO2018/015573 |
| | | Consensus |
| | | CDR-H1 |
| 67 | IRNKANGYTT | WO2018/015573 |
| | | Consensus |
| | | CDR-H2 |
| 68 | ARIGINNGGSLDYWG | WO2018/015573 |
| | | Consensus |
| | | CDR-H3 |
| 69 | QSLLYSENNQDY | WO2018/015573 |
| | | Consensus |
| | | CDR-L1 |
| 70 | GAS | WO2018/015573 |
| | | Consensus |
| | | CDR-L2 |
| 71 | EQTYSYPYT | WO2018/015573 |
| | | Consensus |
| | | CDR-L3 |
| 72 | EVKLLESGGGLVQPGGSMRLSCAASGFTFTDFYMNWIRQ | WO2018/015573 |
| | PAGKAPEWLGLIRNKANGYTTEYNPSVKGRFTISRDNTQN | Consensus |
| | MLYLQMNTLR*EDTATYYCARIGINNGGSLDYWGQGVM | Heavy Chain |
| | VTVSS | Variable Region |
| | | The asterisk (*) in the |
| | | sequence can be any |
| | | amino acid. |
| 73 | DILINQSPASLTVSAGEKVTMSCKSSQSLLYSENNQDYLA | WO2018/015573 |
| | WYQQKPGQFPKLLIYGASNRHTGVPDRFTGSGSGTDFTLT | Consensus |
| | ISSVQAEDLADYYCEQTYSYPYTFGAGTKLELK | Light Chain Variable |
| | | Region |
| 74 | GFTFTDFY | WO2018/015573 |
| | | 14D3 |
| | | CDR-H1 |
| 75 | IRNKTKGYTT | WO2018/015573 |
| | | 14D3 |
| | | CDR-H2 |
| 76 | ARIGVNNGGSLDYWG | WO2018/015573 |
| | | 14D3 |
| | | CDR-H3 |
| 77 | QSLLYSENNQDY | WO2018/015573 |
| | | 14D3 |
| | | CDR-L1 |
| 78 | GAS | WO2018/015573 |
| | | 14D3 |
| | | CDR-L2 |
| 79 | EQTYSYPYT | WO2018/015573 |
| | | 14D3 |
| | | CDR-L3 |
| 80 | EVKLLEFGGGLVQPGGSMRLSCAASGFTFTDFYMNWIRQ | WO2018/015573 |
| | PAGRAPEWLGLIRNKTKGYTTEYNRSVKGRFTISRDNTQN | 14D3 |
| | MLYLQMNSLRPEDTATYYCARIGVNNGGSLDYWGQGVM | Heavy Chain |
| | VTVSS | Variable Region |
| 81 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 14D3 |
| | SVQAEDLADYYCEQTYSYPYTFGAGTKLELK | Light Chain Variable |
| | | Region |
| 82 | GFTFTDFY | WO2018/015573 |
| | | 14D8 |
| | | CDR-H1 |
| 83 | IRNKANGYTT | WO2018/015573 |
| | | 14D8 |
| | | CDR-H2 |
| 84 | ARIGINNGGSLDYWG | WO2018/015573 |
| | | 14D8 |
| | | CDR-H3 |
| 85 | QSLLYSEKNQDY | WO2018/015573 |
| | | 14D8 |
| | | CDR-L1 |
| 86 | GAS | WO2018/015573 |
| | | 14D8 |
| | | CDR-L2 |
| 87 | EQTYSYPYT | WO2018/015573 |
| | | 14D8 |
| | | CDR-L3 |
| 88 | EVKLLESGGGLVQPGGSMRLSCAASGFTFTDFYMNWIRQ | WO2018/015573 |
| | PAGKAPEWLGLIRNKANGYTTVYNPSVKGRFTISRDNTQ | 14D8 |
| | NMLYLQMNTLRGEDTATYYCARIGINNGGSLDYWGQGV | Heavy Chain |
| | MVTVSS | Variable Region |
| 89 | DILINQSPASLTVSTGEKVTMSCRSSQSLLYSEKNQDYLA | WO2018/015573 |
| | WYQQKPGQFPKLLIYGASYRHTGVPDRFTGSGSGTDFTLT | 14D8 |
| | ISSVQAEDLADYYCEQTYSYPYTFGAGTKLELK | Light Chain Variable |
| | | Region |
| 90 | GFTFTDFY | WO2018/015573 |
| | | 7A12 |
| | | CDR-H1 |
| 91 | IRNKANGYTT | WO2018/015573 |
| | | 7A12 |
| | | CDR-H2 |
| 92 | ARIGINNGGSLDYWG | WO2018/015573 |
| | | 7A12 |
| | | CDR-H3 |
| 93 | QSLLYSEKNQDY | WO2018/015573 |
| | | 7A12 |
| | | CDR-L1 |
| 94 | GAS | WO2018/015573 |
| | | 7A12 |
| | | CDR-L2 |
| 95 | EQTYSYPYT | WO2018/015573 |
| | | 7A12 |
| | | CDR-L3 |
| 96 | EVKLLESGGGLVQPGGSMRLSCAASGFTFTDFYMNWIRQ | WO2018/015573 |
| | PAGKAPEWLGLIRNKANGYTTQYNPSVKGRFTISRDNTQ | 7A12 |
| | NMLYLQMNTLRGEDTATYYCARIGINNGGSLDYWGQGV | Heavy Chain |
| | MVTVSS | Variable Region |
| 97 | DILINQSPASLTVSAGEKVTMSCKSSQSLLYSEKNQDYLA | WO2018/015573 |
| | WYQQKPGQSPKLLMYGASYRHTGVPDRFTGSGSGTDFTL | 7A12 |
| | TISSVQAEDLADYYCEQTYSYPYTFGAGTKLELK | Light Chain Variable |
| | | Region |
| 98 | GFTFTDFY | WO2018/015573 |
| | | 8A11 |
| | | CDR-H1 |
| 99 | IRNKTKGYTT | WO2018/015573 |
| | | 8A11 |
| | | CDR-H2 |
| 100 | ARIGVNNGGSLDYWG | WO2018/015573 |
| | | 8A11 |
| | | CDR-H3 |
| 101 | QSLLYSENNQDY | WO2018/015573 |
| | | 8A11 |
| | | CDR-L1 |
| 102 | GAS | WO2018/015573 |
| | | 8A11 |
| | | CDR-L2 |
| 103 | EQTYSYPYT | WO2018/015573 |
| | | 8A11 |
| | | CDR-L3 |
| 104 | EVKLLESGGGLVQPGGSMRLSCAASGFTFTDFYMNWIRQ | WO2018/015573 |
| | PAGKAPEWLGLIRNKTKGYTTEYNTSVKGRFTISRDNTQN | 8A11 |
| | MLYLQMNSLRPEDTATYYCARIGVNNGGSLDYWGQGVM | Heavy Chain |
| | VTVSS | Variable Region |
| 105 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 8A11 |
| | SVQAEDLADYYCEQTYSYPYTFGAGTKLELK | Light Chain Variable |
| | | Region |
| 106 | GFTFTDFY | WO2018/015573 |
| | | 21A3 |
| | | CDR-H1 |
| 107 | IRNKANGYTT | WO2018/015573 |
| | | 21A3 |
| | | CDR-H2 |
| 108 | ARIGINNGGSLDYWG | WO2018/015573 |
| | | 21A3 |
| | | CDR-H3 |
| 109 | QSLLYSEKNQDY | WO2018/015573 |
| | | 21A3 |
| | | CDR-L1 |
| 110 | GAS | WO2018/015573 |
| | | 21A3 |
| | | CDR-L2 |
| 111 | EQTYSYPYT | WO2018/015573 |
| | | 21A3 |
| | | CDR-L3 |
| 112 | EVKLLESGGGLVQPGGSMRLSCAASGFTFTDFYMNWIRQ | WO2018/015573 |
| | PAGKAPEWLGLIRNKANGYTTQYNPSVKGRFTISRDNTQ | 21A3 |
| | NMLYLQMNTLRGEDTATYYCARIGINNGGSLDYWGQGV | Heavy Chain |
| | MVTVSS | Variable Region |
| 113 | DILINQSPASLTVSAGEKVTMSCKSSQSLLYSEKNQDYLA | WO2018/015573 |
| | WYQQKPGQSPKLLMYGASYRHTGVPDRFTGSGSGTDFTL | 21A3 |
| | TISSVQAEDLADYYCEQTYSYPYTFGAGTKLELK | Light Chain Variable |
| | | Region |
| 114 | GFTFTDFY | WO2018/015573 |
| | | 10C3 |
| | | CDR-H1 |
| 115 | IRNKTKGYTT | WO2018/015573 |
| | | 10C3 |
| | | CDR-H2 |
| 116 | ARIGTNNGGSLDYWG | WO2018/015573 |
| | | 10C3 |
| | | CDR-H3 |
| 117 | QSLLYSENNQDY | WO2018/015573 |
| | | 10C3 |
| | | CDR-L1 |
| 118 | GAS | WO2018/015573 |
| | | 10C3 |
| | | CDR-L2 |
| 119 | EQTYSYPYT | WO2018/015573 |
| | | 10C3 |
| | | CDR-L3 |
| 120 | EVKLLESGGGLVQPGGSMRLSCAASGFTFTDFYMNWIRQ | WO2018/015573 |
| | PAGETPEWLGLIRNKTKGYTTEYNPSVKGRFTISRDNTQN | 10C3 |
| | MLYLQMNSLRPEDTATYYCARIGTNNGGSLDYWGQGVM | Heavy Chain |
| | VTVSS | Variable Region |
| 121 | DILIIQSPASLTVSAGARVTMSCKSSQSLLYSENNQDYLAW | WO2018/015573 |
| | YQQKPGQFPKLLIYGASNRHTGVPDRFTGSGSGTDFTLTIS | 10C3 |
| | SVQAEDLADYYCEQTYSYPYTFGAGTKLELK | Light Chain Variable |
| | | Region |
| 122 | GFTFTDFY | WO2018/015573 |
| | | 18F9 |
| | | CDR-H1 |
| 123 | IRNKVNGYRT | WO2018/015573 |
| | | 18F9 |
| | | CDR-H2 |
| 124 | ARIGINNGGSLDYWG | WO2018/015573 |
| | | 18F9 |
| | | CDR-H3 |
| 125 | QSLLYSENNQDY | WO2018/015573 |
| | | 18F9 |
| | | CDR-L1 |
| 126 | GAS | WO2018/015573 |
| | | 18F9 |
| | | CDR-L2 |
| 127 | EQTYSYPYT | WO2018/015573 |
| | | 18F9 |
| | | CDR-L3 |
| 128 | EVKLLESGGGLVQPGGSMRLSCVVSGFTFTDFYMNWIRQ | WO2018/015573 |
| | AAGKAPEWLGLIRNKVNGYRTEYNPSVKGRFTISRDNIQN | 18F9 |
| | MLYLQMNTLRAEDTATYYCARIGINNGGSLDYWGQGVM | Heavy Chain |
| | VTVSS | Variable Region |
| 129 | DILINQSPASLTVSAGEKVTMSCKSSQSLLYSENNQDYLA | WO2018/015573 |
| | WYQQKPGQFPKLLIYGASNRHTGVPDRFTGSGSGTDFTLT | 18F9 |
| | ISSVQAEDLADYYCEQTYSYPYTFGAGTKLELK | Light Chain Variable |
| | | Region |
| 130 | GFTFTDFY | WO2018/015573 |
| | | 15C5 |
| | | CDR-H1 |
| 131 | IRNKAYGYTT | WO2018/015573 |
| | | 15C5 |
| | | CDR-H2 |
| 132 | ARIGINYGGSLDYWG | WO2018/015573 |
| | | 15C5 |
| | | CDR-H3 |
| 133 | QSLLYSESNQDY | WO2018/015573 |
| | | 15C5 |
| | | CDR-L1 |
| 134 | GAS | WO2018/015573 |
| | | 15C5 |
| | | CDR-L2 |
| 135 | EQTYSYPYT | WO2018/015573 |
| | | 15C5 |
| | | CDR-L3 |
| 136 | EVKLLESGGGLVQPGGSMRLSCAASGFTFTDFYMNWIRQ | WO2018/015573 |
| | PAGKAPEWLGLIRNKAYGYTTEYNPSVKGRFTISRDNTQD | 15C5 |
| | MLYLQMNTLRAEDTATYYCARIGINYGGSLDYWGQGVM | Heavy Chain |
| | VTVSS | Variable Region |
| 137 | DILINQSPASLTVSAGEKVTVSCKSSQSLLYSESNQDYLAW | WO2018/015573 |
| | YQQKPGQFPKLLIYGASYRHTGVPDRFTGSGSGTDFTLTIS | 15C5 |
| | SVQAEDLAHYYCEQTYSYPYTFGAGTKLELK | Light Chain Variable |
| | | Region |
| 138 | GFTFTDFY | WO2018/015573 |
| | | 1G6 |
| | | CDR-H1 |
| 139 | IRNKANGFTT | WO2018/015573 |
| | | 1G6 |
| | | CDR-H2 |
| 140 | ARIGINNGGSLDYWG | WO2018/015573 |
| | | 1G6 |
| | | CDR-H3 |
| 141 | QSLLYSENKQDY | WO2018/015573 |
| | | 1G6 |
| | | CDR-L1 |
| 142 | GAS | WO2018/015573 |
| | | 1G6 |
| | | CDR-L2 |
| 143 | EQTYSYPYT | WO2018/015573 |
| | | 1G6 |
| | | CDR-L3 |
| 144 | EVKLLESGGGLVQPGGSLRLSCVASGFTFTDFYMNWIRQP | WO2018/015573 |
| | AGKAPEWLGLIRNKANGFTTEYNPSVKGRFTISRDNTQH | 1G6 |
| | MLYLQMNTLRAEDTATYYCARIGINNGGSLDYWGQGVM | Heavy Chain |
| | VTVSS | Variable Region |
| 145 | DILINQSPASLTVSTGEKVTMSCKSSQSLLYSENKQDYLA | WO2018/015573 |
| | WYQQKPGQFPKLLIYGASNRHTGVPDRFTGSGSGTDFTLT | 1G6 |
| | INIVQAEDLADYYCEQTYSYPYTFGAGTKLELK | Light Chain Variable |
| | | Region |

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

Antibody Fragments

Certain aspects of the present disclosure relate to antibody fragments that bind to one or more of human TREM2, a naturally occurring variant of human TREM2, and a disease variant of human TREM2. In some embodiments, the antibody fragment is an Fab, Fab′, Fab′-SH, F(ab′)2, Fv or scFv fragment.

Antibody Frameworks

Any of the antibodies described herein further include a framework. In some embodiments, the framework is a human immunoglobulin framework. For example, in some embodiments, an antibody (e.g., an anti-TREM2 antibody) comprises HVRs as in any of the above embodiments and further comprises an acceptor human framework, e.g., a human immunoglobulin framework or a human consensus framework. Human immunoglobulin frameworks may be part of the human antibody, or a non-human antibody may be humanized by replacing one or more endogenous frameworks with human framework region(s). Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

Antibody Preparation

Anti-TREM2 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, library derived antibodies, antibodies having modified effector functions, fusion proteins containing an antibody portion, and any other modified configuration of the immunoglobulin molecule that includes an antigen recognition site, such as an epitope having amino acid residues of a TREM2 protein of the present disclosure, including glycosylation variants of antibodies, amino acid sequence variants of antibodies, and covalently modified antibodies. The anti-TREM2 antibodies may be human, murine, rat, or of any other origin (including chimeric or humanized antibodies).

(1) Polyclonal Antibodies

Polyclonal antibodies, such as anti-TREM2 polyclonal antibodies, are generally raised in animals by multiple subcutaneous (sc) or intraperitoneal (ip) injections of the relevant antigen and an adjuvant. It may be useful to conjugate the relevant antigen (e.g., a purified or recombinant TREM2 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant-cell culture as protein fusions. Also, aggregating agents such as alum are suitable to enhance the immune response.

(2) Monoclonal Antibodies

Monoclonal antibodies, such as anti-TREM2 monoclonal antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the anti-TREM2 monoclonal antibodies may be made using the hybridoma method first described by Köhler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant TREM2 protein of the present disclosure). Alternatively, lymphocytes may be immunized in vitro. Lymphocytes then are fused with an immortal cell line, such as myeloma cells, using a suitable fusing agent, such as polyethylene glycol, to form a hybridoma cell (Goding, Monoclonal Antibodies: Principles and Practice, pp. 59-103 (Academic Press, 1986)).

The culture medium in which the hybridoma cells are cultured can be assayed for the presence of monoclonal antibodies directed against the desired antigen (e.g., a TREM2 protein of the present disclosure), e.g., as determined by immunoprecipitation or by an in vitro binding assay, such as radioimmunoassay (RIA) or enzyme-linked assay (ELISA). Such techniques and assays are known in the in art.

After hybridoma cells are identified that produce antibodies of the desired specificity, affinity, and/or activity, the clones may be subcloned, and monoclonal antibodies secreted by the subclones may be separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-TREM2 monoclonal antibodies may also be made by recombinant DNA methods, e.g., as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host-cells such as E. coli cells, simian COS cells, Chinese hamster ovary (CHO) cells, or myeloma cells that do not otherwise produce immunoglobulin protein, in order to synthesize monoclonal antibodies in such recombinant host-cells. Review articles on recombinant expression in bacteria of DNA encoding the antibody include Skerra et al., Curr. Opin. Immunol., 5:256-262 (1993) and Phickthun, Immunol. Rev. 130:151-188 (1992).

In certain embodiments, anti-TREM2 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio Technology, 10:779-783 (1992)), as well as combinatorial infection and in vivo recombination as a strategy for constructing very large phage libraries (Waterhouse et al., Nucl. Acids Res., 21:2265-2266 (1993)).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(3) Humanized Antibodies

Anti-TREM2 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321: 522-525 (1986); Riechmann et al., Nature 332: 323-329 (1988) and Presta, Curr. Opin. Struct. Biol. 2: 593-596 (1992).

Certain methods for humanizing non-human anti-TREM2 antibodies are known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies may impact immunogenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Humanized antibodies preferably retain high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., TREM2 proteins of the present disclosure), is achieved. In general, the CDR residues are directly and most substantially involved in influencing antigen binding.

Various forms of the humanized anti-TREM2 antibody are contemplated. For example, the humanized anti-TREM2 antibody may be an antibody fragment, such as an Fab, or an intact antibody, such as an intact IgG1 antibody.

(4) Antibody Fragments

In certain embodiments there are advantages to using anti-TREM2 antibody fragments, rather than whole anti-TREM2 antibodies. In some embodiments, smaller fragment sizes allow for rapid clearance and better brain penetration.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host-cells, for example, using nucleic acids encoding anti-TREM2 antibodies of the present disclosure. Fab, Fv and scFv antibody fragments can all be expressed in and secreted from E. coli, thus allowing the straightforward production of large amounts of these fragments. Anti-TREM2 antibody fragments can also be isolated from the antibody phage libraries as discussed above. Alternatively, Fab′-SH fragments can be directly recovered from E. coli and chemically coupled to form F(ab′)₂ fragments (Carter et al., Bio Technology 10:163-167 (1992)). According to another approach, F(ab′)2 fragments can be isolated directly from recombinant host-cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-TREM2 antibody fragment may also be a “linear antibody,” e.g., as described in U.S. Pat. No. 5,641,870. Such linear antibody fragments may be monospecific or bispecific.

(5) Bispecific and Polyspecific Antibodies

Bispecific antibodies (BsAbs) are antibodies that have binding specificities for at least two different epitopes, including those on the same or another protein (e.g., one or more TREM2 proteins of the present disclosure). Alternatively, one part of a BsAb can be armed to bind to the target TREM2 antigen, and another can be combined with an arm that binds to a second protein. Such antibodies can be derived from full-length antibodies or antibody fragments (e.g., F(ab′)₂ bispecific antibodies).

(6) Effector Function Engineering

It may also be desirable to modify an anti-TREM2 antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce Antibody-dependent cell-mediated cytotoxicity. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40: 585-593 (2003); Reddy et al., J. Immunology 164:1925-1933 (2000). In other embodiments, it may also be desirable to modify an anti-TREM2 antibody of the present disclosure to modify effector function to increase finding selectivity toward the ITIM-containing FcgRIIb (CD32b) to increase clustering of TREM2 antibodies on adjacent cells without activating humoral responses including antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

(7) Other Amino Acid Sequence Modifications

Amino acid sequence modifications of anti-TREM2 antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a TREM2 protein of the present disclosure). The amino acid changes also may alter post-translational processes of the antibody, such as changing the number or position of glycosylation sites.

A useful method for identification of certain residues or regions of the anti-TREM2 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino- (“N”) and/or carboxy- (“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table C below under the heading of “preferred substitutions”. If such substitutions result in a change in biological activity, then more substantial changes, denominated “exemplary substitutions” in Table C, or as further described below in reference to amino acid classes, may be introduced and the products screened.

TABLE C
Amino Acid Substitutions

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-TREM2 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a TREM2 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development. Affinity maturation may also be performed by employing a yeast presentation technology such as that disclosed in, for example, WO2009/036379A2; WO2010105256; WO2012009568; and Xu et al., Protein Eng. Des. Sel., 26(10): 663-70 (2013).

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

(8) Other Antibody Modifications

Anti-TREM2 antibodies of the present disclosure, or antibody fragments thereof, can be further modified to contain additional non-proteinaceous moieties that are known in the art and readily available, or to contain different types of drug conjugates that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

Drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue, OncLive Jul. 5, 2012; ADC Review on antibody-drug conjugates; and Ducry et al., (2010). Bioconjugate Chemistry 21 (1): 5-13).

(9) Binding Assays and Other Assays

Anti-TREM2 antibodies of the present disclosure may be tested for antigen binding activity, e.g., by known methods such as ELISA, Western blot, etc.

Detailed exemplary methods for mapping an epitope to which an antibody binds are provided in Morris (1996) “Epitope Mapping Protocols,” in Methods in Molecular Biology vol. 66 (Humana Press, Totowa, N.J.).

### Nucleic Acids, Vectors, and Host Cells

Anti-TREM2 antibodies of the present disclosure may be produced using recombinant methods and compositions, e.g., as described in U.S. Pat. No. 4,816,567. In some embodiments, isolated nucleic acids having a nucleotide sequence encoding any of the anti-TREM2 antibodies of the present disclosure are provided. Such nucleic acids may encode an amino acid sequence containing the VL and/or an amino acid sequence containing the VH of the anti-TREM2 antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-TREM2 antibody of the present disclosure are provided. In some embodiments, the method includes culturing a host cell of the present disclosure containing a nucleic acid encoding the anti-TREM2 antibody, under conditions suitable for expression of the antibody. In some embodiments, the antibody is subsequently recovered from the host cell (or host cell culture medium).

For recombinant production of an anti-TREM2 antibody of the present disclosure, a nucleic acid encoding the anti-TREM2 antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-TREM2 antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-TREM2 antibody of the present disclosure.

Suitable host cells for cloning or expression of antibody-encoding vectors include prokaryotic or eukaryotic cells. For example, anti-TREM2 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Pharmaceutical Compositions and Formulations

Provided herein are pharmaceutical compositions comprising an anti-TREM2 antibody of the present disclosure and a pharmaceutically acceptable carrier. In some embodiments, provided herein are pharmaceutical compositions comprising the anti-TREM2 antibody of the present disclosure having the desired degree of purity in a physiologically acceptable carrier, excipient or stabilizer (Remington's Pharmaceutical Sciences (1990) Mack Publishing Co., Easton, Pa.). Acceptable carriers, excipients, or stabilizers are nontoxic to recipients at the dosages and concentrations employed.

In various embodiments, pharmaceutical compositions comprising an anti-TREM2 antibody are provided in formulations with a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Pharmaceutical Dosages and Administration

An anti-TREM2 antibody provided herein can be administered by any suitable means, including parenteral, intrapulmonary, intranasal, intralesional administration, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous administration. In some embodiments, the administration is subcutaneous. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of an anti-TREM2 antibody will depend on the type of disease to be treated, the particular antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

### Kits/Articles of Manufacture

The present disclosure also provides kits containing an isolated antibody of the present disclosure (e.g., an anti-TREM2 antibody described herein), or a functional fragment thereof. Kits of the present disclosure may include one or more containers comprising a purified antibody of the present disclosure. In some embodiments, the kits further include instructions for use in accordance with the methods of this disclosure. In some embodiments, these instructions comprise a description of administration of the isolated antibody of the present disclosure (e.g., an anti-TREM2 antibody described herein) to prevent, reduce risk, or treat an individual having a disease, disorder, or injury selected from pediatric-onset leukoencephalopathy; adult-onset leukoencephalopathy with axonal spheroids and pigmented glia (ALSP); leukoencephalopathy, diffuse hereditary, with spheroids; adult-onset leukodystrophy with neuroaxonal spheroids; autosomal dominant leukoencephalopathy with neuroaxonal spheroids; hereditary diffuse leukoencephalopathy with axonal spheroids (HDLS); neuroaxonal leukodystrophy; pigmentary orthochromatic leukodystrophy; and familial pigmentary orthochromatic leukoencephalopathy (POLD), according to any methods of this disclosure.

In some embodiments, the instructions comprise a description of how to detect TREM2, for example in an individual, in a tissue sample, or in a cell. The kit may further comprise a description of selecting an individual suitable for treatment based on identifying whether that individual has the disease and the stage of the disease.

In some embodiments, the kits may further include another antibody of the present disclosure (e.g., at least one antibody that specifically binds to an inhibitory checkpoint molecule, at least one antibody that specifically binds to an inhibitory cytokine, and/or at least one agonistic antibody that specifically binds to a stimulatory checkpoint protein) and/or at least one stimulatory cytokine. In some embodiments, the kits may further include instructions for using the antibody and/or stimulatory cytokine in combination with an isolated antibody of the present disclosure (e.g., an anti-TREM2 antibody described herein), instructions for using the isolated antibody of the present disclosure in combination with an antibody and/or stimulatory cytokine, or instructions for using an isolated antibody of the present disclosure and an antibody and/or stimulatory cytokine, according to any methods of this disclosure.

The instructions generally include information as to dosage, dosing schedule, and route of administration for the intended treatment. The containers may be unit doses, bulk packages (e.g., multi-dose packages) or sub-unit doses. Instructions supplied in the kits of the present disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a disease of the present disclosure. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is an isolated antibody of the present disclosure (e.g., an anti-TREM2 antibody described herein). The container may further comprise a second pharmaceutically active agent.

Kits may optionally provide additional components such as buffers and interpretive information. Normally, the kit comprises a container and a label or package insert(s) on or associated with the container.

The present disclosure will be more fully understood by reference to the following Examples. They should not, however, be construed as limiting the scope of the present disclosure. All citations throughout the disclosure are hereby expressly incorporated by reference.

### Biomarkers

In certain embodiments, CSF1R levels are used as a biomarker for activity of a TREM2 antibody as described herein. In certain embodiments, the TREM2 antibody that is administered to a subject significantly induces CSF1R expression or increases CSF1R levels, relative to an untreated subject or a subject treated with a control antibody or placebo. In certain embodiments, the TREM2 antibody increases CSF1R RNA or protein levels. In certain embodiments, the TREM2 antibody increases CSF1R RNA levels, e.g., by 10%, 20%, 25%, 30%, 35%, 40%, 45%, 50%, 60%, 70%, 80, 90% or 100%. In certain embodiments, the TREM2 antibody increases CSF1R protein levels, e.g., by 10%, 20%, 25%, 30%, 35%, 40%, 45%, 50%, 60%, 70%, 80, 90% or 100%. In certain embodiments, CSF1R RNA or protein levels are increased in the brain, for example, as detected in cerebrospinal fluid. In certain embodiments, CSF1R RNA or protein levels are increased in the frontal cortex. In certain embodiments, CSF1R RNA or protein levels are increased in the hippocampus. In certain embodiments, CSF1R RNA or protein levels are increased in the plasma. In certain embodiments, CSF1R RNA or protein levels are used as a biomarker for the activity of an anti-TREM2 agonistic antibody. In certain embodiments, an anti-TREM2 agonistic antibody increases CSF1R RNA levels, e.g., by 10%, 20%, 25%, 30%, 35%, 40%, 45%, 50%, 60%, 70%, 80, 90% or 100%. In certain embodiments, an anti-TREM2 agonistic antibody increases CSF1R protein levels, e.g., by 10%, 20%, 25%, 30%, 35%, 40%, 45%, 50%, 60%, 70%, 80, 90% or 100%. In certain embodiments, an anti-TREM2 agonistic antibody increases CSF1R RNA or protein levels in the brain, for example, as detected in cerebrospinal fluid. In certain embodiments, an anti-TREM2 agonistic antibody increases CSF1R RNA or protein levels in the frontal cortex. In certain embodiments, an anti-TREM2 agonistic antibody increases CSF1R RNA or protein levels in the hippocampus. In certain embodiments, an anti-TREM2 agonistic antibody increases CSF1R RNA or protein levels in the plasma.

In certain embodiments, CSF1R RNA or protein levels are used as a biomarker for AL2p-58 huIgG1 PSEG activity. In certain embodiments, AL2p-58 huIgG1 PSEG increases CSF1R RNA levels, e.g., by 10%, 20%, 25%, 30%, 35%, 40%, 45%, 50%, 60%, 70%, 80, 90% or 100%. In certain embodiments, AL2p-58 huIgG1 PSEG increases CSF1R protein levels, e.g., by 10%, 20%, 25%, 30%, 35%, 40%, 45%, 50%, 60%, 70%, 80, 90% or 100%. In certain embodiments, AL2p-58 huIgG1 PSEG increases CSF1R RNA or protein levels in the brain, for example, as detected in cerebrospinal fluid. In certain embodiments AL2p-58 huIgG1 PSEG increases CSF1R RNA or protein levels in the frontal cortex. In certain embodiments, AL2p-58 huIgG1 PSEG increases CSF1R RNA or protein levels in the hippocampus. In certain embodiments, AL2p-58 huIgG1 PSEG increases CSF1R RNA or protein levels in the plasma.

In certain embodiments, CSF1R RNA or protein levels are used as a biomarker for AL2p-47 huIgG1 activity. In certain embodiments, AL2p-47 huIgG1 increases CSF1R RNA levels, e.g., by 10%, 20%, 25%, 30%, 35%, 40%, 45%, 50%, 60%, 70%, 80, 90% or 100%. In certain embodiments, AL2p-47 huIgG1 increases CSF1R protein levels, e.g., by 10%, 20%, 25%, 30%, 35%, 40%, 45%, 50%, 60%, 70%, 80, 90% or 100%. In certain embodiments, AL2p-47 huIgG1 increases CSF1R RNA or protein levels in the brain, for example, as detected in cerebrospinal fluid. In certain embodiments AL2p-47 huIgG1 increases CSF1R RNA or protein levels in the frontal cortex. In certain embodiments, AL2p-47 huIgG1 increases CSF1R RNA or protein levels in the hippocampus. In certain embodiments, AL2p-47 huIgG1 increases CSF1R RNA or protein levels in the plasma.

In some embodiments of the methods of treatment described herein, the method comprises a step of measuring the level of CSF1R in a sample from the individual. The sample may be from the blood of the cerebrospinal fluid of the individual. The RNA level of CSF1R or the protein level of CSF1R may be measured by any technique known to one of skill in the art. In some embodiments, the level of CSF1R is measured before and after administering the anti-TREM2 antibody and the difference in the level of CSF1R is calculated. In some embodiments, the anti-TREM2 antibody is considered to be active in the individual if the level of CSF1R increases after administration of the anti-TREM2 antibody. In some embodiments, the anti-TREM2 antibody is considered to not be active in the individual if the level of CSF1R does not increase after administration of the anti-TREM2 antibody.

The present disclosure provides a method of monitoring the treatment of an individual being administered an anti-TREM2 antibody comprising measuring the level of CSF1R in a sample from the individual before and after the individual has received one or more doses of an anti-TREM2 antibody. In some embodiments, the method is monitoring the treatment of an individual being administered AL2p-58 huIgG1 PSEG. In some embodiments, the method is monitoring the treatment of an individual being administered AL2p-47 huIgG1. In some embodiments, the sample is from the cerebrospinal fluid of the individual or the blood of the individual. In some embodiments, the method further comprises a step of assessing the activity of the anti-TREM2 antibody in the individual based on the level of CSF1R in the sample. For example, in some embodiments, the anti-TREM2 antibody is considered to be active in the individual if the level of CSF1R increases after administration of the anti-TREM2 antibody, and in some embodiments, the anti-TREM2 antibody is considered to not be active in the individual if the level of CSF1R does not increase after administration of the anti-TREM2 antibody.

### EXAMPLES

### Example 1: Human Macrophage Cell Viability Following CSF1 Withdrawal

To evaluate the ability of an anti-TREM2 agonistic antibody to sustain survival of human macrophages after CSF1 withdrawal, human monocytes were isolated from whole blood using RosetteSep Human Monocyte Enrichment Protocol (Stem Cell Technologies). To prepare human monocyte derived macrophages, monocytes were counted and plated in complete RPMI media (RPMI supplemented with Glutamax, penicillin/streptomycin, non-essential amino acids, sodium-pyruvate, and 10% heat inactivated fetal bovine serum) and 50 ng/ml M-CSF (Peprotech). After 6 days, differentiated monocytes (macrophages) were harvested and plated onto 96-well plates at a density of 1.0×10³ cells/well in complete RPMI media with M-CSF. Cells were allowed to recover overnight. On Day 7, cell media was replaced with serum-free RPMI media and cells were treated with M-CSF only (50 ng/mL), IgG1 (10 μg/mL), AL2p-58 huIgG1 PSEG (1 μg/mL), or AL2p-58 huIgG1 PSEG (10 μg/mL). Cell viability was quantified using the CellTiter-Glo luminescent viability assay (Promega) on each subsequent day post M-CSF withdrawal.

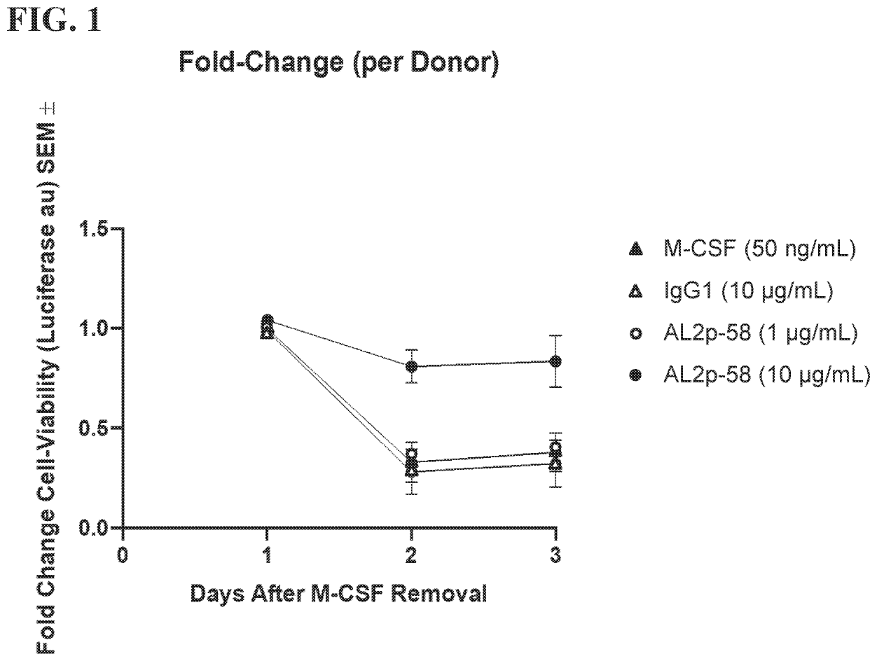

As shown in FIG. 1, human macrophages treated with AL2p-58 huIgG1 PSEG (10 μg/mL) had significantly increased cell viability compared to cells treated with M-CSF alone (50 ng/mL), IgG1 (10 μg/mL), or AL2p-58 huIgG1 PSEG (1 μg/mL). Specifically, AL2p-58 huIgG1 PSEG treatment at 10 μg/mL significantly improved cell viability compared to M-CSF alone.

### Example 2: Human Macrophage Cell Viability and Survival Following CSF1R Inhibition

The results presented in Example 1 demonstrated that treatment with an anti-TREM2 agonistic antibody can sustain the survival of human macrophages after withdrawal of M-CSF1. However, M-CSF1 is only one ligand of the receptor CSF1R. In the present example, experiments were conducted to investigate the effect of anti-TREM2 agonistic antibody on viability of human macrophages when the receptor CSF1R itself is inhibited.

To evaluate the ability of an anti-TREM2 antibody to sustain survival of human macrophages after CSF1R inhibition, the ability of AL2p-58 huIgG1 PSEG to enhance cell-viability and survival in the presence of CSF1R inhibitor PLX3397 was tested (DeNardo et al., Cancer Discov (2011) 1(1):54-67. 22039576t; Peng, et al., J. of Exp Canc Res (2019) 38(1):372. PMID: 31438996). Similar to CSF1 withdrawal experiments, human derived macrophages were plated onto 96-well plates on Day 6 and treated with IgG1, PLX3397 (30 nM), AL2p-58 huIgG1 PSEG (10 μg/mL) or a combination of PLX3397 and AL2p-58 huIgG1 PSEG on Day 7, all in complete RPMI media. Cell viability was quantified using the CellTiter-Glo luminescent viability assay (Promega) on each subsequent day.

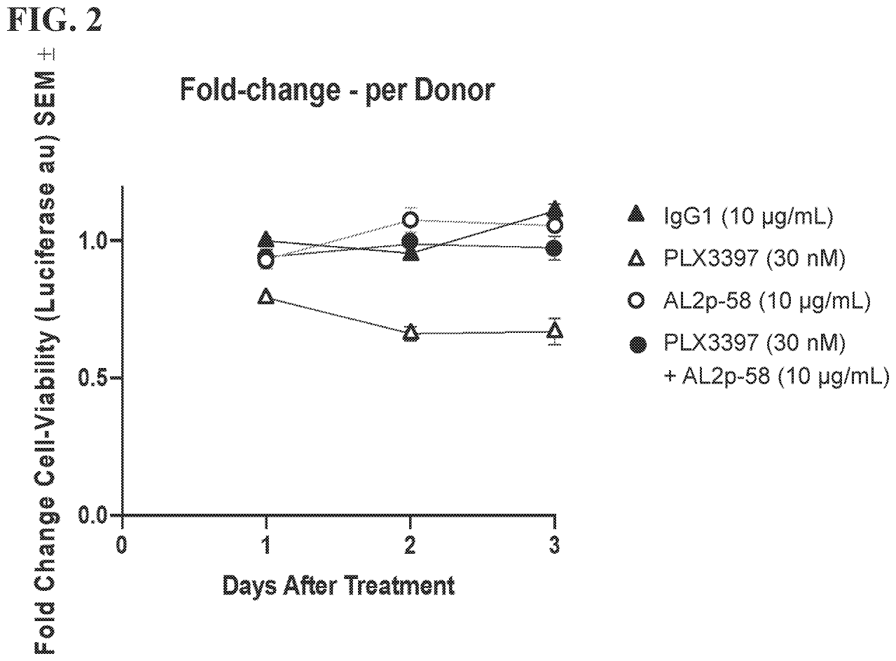

As shown in FIG. 2, treatment with AL2p-58 huIgG1 PSEG sustained survival of human macrophages in the presence of CSF1R inhibition. Specifically, the data presented in FIG. 2 demonstrated that human macrophages treated with PLX3397 (a CSF1_R inhibitor) had reduced cell viability. However, there was a significant improvement in cell viability when cells treated with PLX3397 were also treated with AL2p-58 huIgG1 PSEG. Further, the data indicated that human macrophages treated with both PLX3397 and AL2p-58 huIgG1 PSEG had a similar level of cell viability to cells that were not subjected to CSF1R inhibition (e.g. treatment with IgG1 or treatment with AL2p-58 huIgG1 PSEG alone).

### Example 3: An Anti-TREM2 Agonistic Antibody Increases Expression of CSF1R Protein in Non-Human Primates

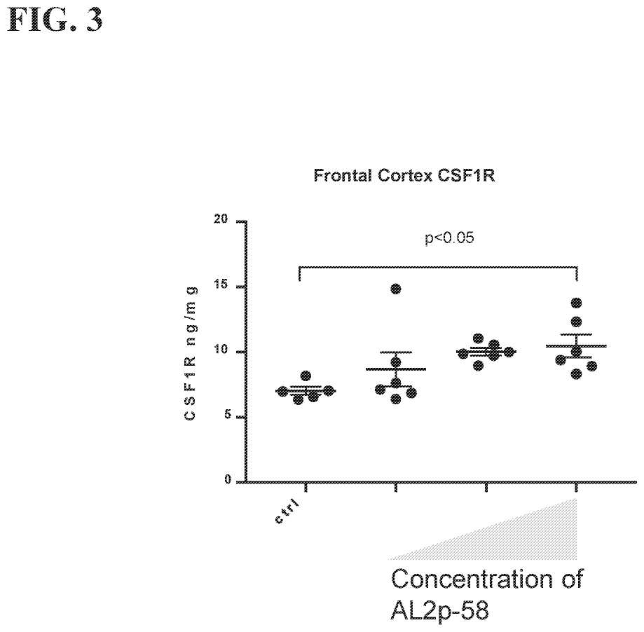

Non-human primates were treated with a control IgG or with increasing concentrations of AL2p-58 huIgG1 PSEG antibody. The level of CSF1R protein in samples from the frontal cortex was measured. As shown in FIG. 3, AL2p-58 huIgG1 PSEG treatment increased CSF1R protein expression in the frontal cortex compared to the control treatment. The highest concentration of AL2p-58 huIgG1 PSEG, which was 12.5× higher than the lowest concentration of AL2p-58 huIgG1 PSEG used in the study, resulted in an increase in CSFR1 levels that was statistically significant compared to control as indicated. Compared to the frontal cortex, increased CSFR1 in the hippocampus was observed but not consistently in non-human primates treated with AL2p-58 huIgG1 PSEG in a comparable study, indicating that AL2p-58 huIgG1 PSEG may have more of an effect on CSF1R levels in certain parts of the brain relative to others.

### Example 4: Anti-TREM2 Antibody Increases CSF1R Levels in the Frontal Cortex and Hippocampus of Non-Human Primates

This Example describes the results of experiments that evaluated the effect of AL2p-58 huIgG1 on the levels of CSF1R protein in the frontal cortex and in the hippocampus of non-human primates (cynomolgus monkeys). AL2p-58 huIgG1 is a variant of anti-TREM2 antibody AL2p-58 huIgG1 PSEG having an Fc comprising wild-type IgG1.

Cynomolgus monkeys were administered weekly doses of control or anti-TREM2 antibody AL2p-58 huIgG1 by intravenous injection for a total of five doses (N=5 per dose group). Forty-eight hours after the 5^{th} dose, brain tissue was harvested and corresponding lysates were analyzed for CSF1R protein expression.

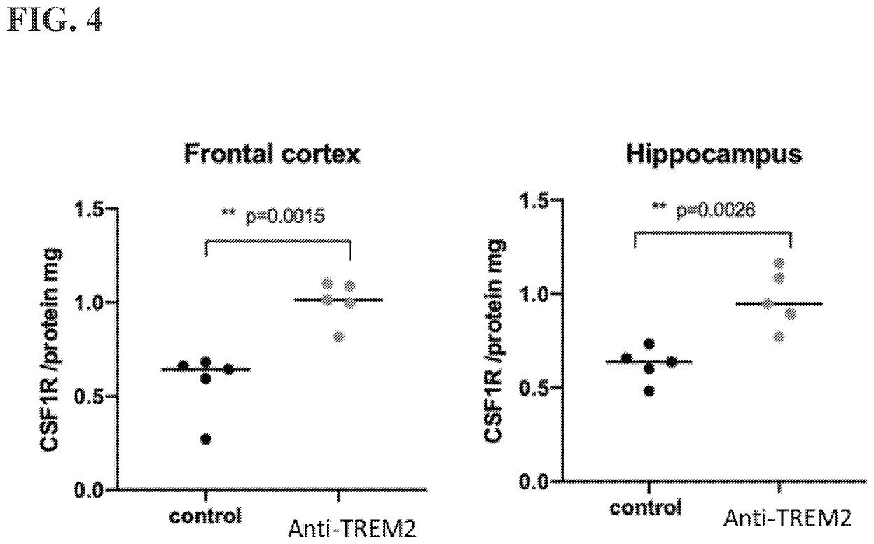

As shown in FIG. 4, CSF1R protein levels in the frontal cortex and in the hippocampus of non-human primates significantly increased following administration of anti-TREM2 antibody AL2p-58 huIgG1 as compared to control-treated animals.

What is claimed is:

1. A method of treating or preventing a CSF1R-deficient disease comprising administering to an individual in need thereof a therapeutically effective amount of an antibody that binds to a TREM2 protein, wherein the antibody is an agonist and wherein the antibody induces one or more TREM2 activities.

2. The method of claim 1, wherein the antibody enhances one or more TREM2 activities induced by binding of one or more TREM2 ligands to the TREM2 protein.

3. The method of claim 2, wherein the antibody enhances the one or more TREM2 activities without blocking binding of the one or more TREM2 ligands to the TREM2 protein.

4. The method of claim 2 or claim 3, wherein the antibody enhances binding of the one or more TREM2 ligands to the TREM2 protein.

5. The method of any one of claims 3-4, wherein the one or more TREM2 ligands are selected from the group consisting of E. coli cells, apoptotic cells, nucleic acids, anionic lipids, anionic lipids, APOE, APOE2, APOE3, APOE4, anionic APOE, anionic APOE2, anionic APOE3, anionic APOE4, lipidated APOE, lipidated APOE2, lipidated APOE3, lipidated APOE4, zwitterionic lipids, negatively charged phospholipids, phosphatidylserine, sulfatides, phosphatidylcholin, sphingomyelin, membrane phospholipids, lipidated proteins, proteolipids, lipidated peptides, lipidated amyloid beta peptide, and any combination thereof.

6. The method of any one of claims 2-5, wherein the antibody enhances the one or more TREM2 activities in the absence of cell surface clustering of TREM2.

7. The method of any one of claims 2-5, wherein the antibody enhances the one or more TREM2 activities by inducing or retaining cell surface clustering of TREM2.

8. The method of any one of claims 1-7, wherein the TREM2 protein is a mammalian protein or a human protein.

9. The method of claim 8, wherein the TREM2 protein is a wild-type protein, a naturally occurring variant, or a disease variant.

10. The method of any one of claims 1-9, wherein the one or more TREM2 activities that are induced or enhanced by the antibody are selected from the group consisting of:
a. TREM2 binding to DAP12;
b. DAP12 phosphorylation;
c. activation of Syk kinase;
d. modulation of one or more pro-inflammatory mediators selected from the group consisting of IFN-β, IL-1α, IL-1β, TNF-α, IL-6, IL-8, CRP, CD86, MCP-1/CCL2, CCL3, CCL4, CCL5, CCR2, CXCL-10, Gata3, IL-20 family members, IL-33, LIF, IFN-gamma, OSM, CNTF, CSF-1, OPN, CD11c, GM-CSF, IL-11, IL-12, IL-17, IL-18, and IL-23, optionally wherein the modulation occurs in one or more cells selected from the group consisting of macrophages, M1 macrophages, activated M1 macrophages, M2 macrophages, dendritic cells, monocytes, osteoclasts, Langerhans cells of skin, Kupffer cells, and microglial cells;
e. recruitment of Syk to a DAP12/TREM2 complex;
f. increasing activity of one or more TREM2-dependent genes, optionally wherein the one or more TREM2-dependent genes comprise nuclear factor of activated T-cells (NFAT) transcription factors;
g. increased survival of dendritic cells, macrophages, M1 macrophages, activated M1 macrophages, M2 macrophages, monocytes, osteoclasts, Langerhans cells of skin, Kupffer cells, microglia, M1 microglia, activated M1 microglia, and M2 microglia, or any combination thereof;
h. modulated expression of one or more stimulatory molecules selected from the group consisting of CD83, CD86 MHC class II, CD40, and any combination thereof, optionally wherein the CD40 is expressed on dendritic cells, monocytes, macrophages, or any combination thereof, and optionally wherein the dendritic cells comprise bone marrow-derived dendritic cells;
i. increasing memory; and
j. reducing cognitive deficit.

11. The method of any one of claims 1-10, wherein the antibody promotes survival of macrophages cultured in the absence of CSF1.

12. The method of any one of claims 1-11, wherein the antibody decreases plasma levels of soluble TREM2 in vivo.

13. The method of any one of claims 1-12, wherein the antibody blocks cleavage of TREM2.

14. The method of any one of claims 1-13, wherein the antibody induces expression of CSF1R or increases levels of CSF1R in the individual compared to an untreated individual or an individual treated with a control antibody.

15. The method of claim 14, wherein the induction of expression of CSF1R or the increase in level of CSF1R occurs in the brain of the individual.

16. The method of any one of claims 1-15, wherein the method comprises a step of measuring the level of CSF1R in a sample from the individual.

17. The method of any one of claims 1-16, wherein the antibody is a murine antibody, a humanized antibody, a bispecific antibody, a multivalent antibody, a conjugated antibody, or a chimeric antibody.

18. The method of any one of claims 1-17, wherein the antibody is a monoclonal antibody.

19. The method of any one of claims 1-18, wherein the antibody binds to one or more amino acids within amino acid residues 124-153 of SEQ ID NO: 1, or amino acid residues on a TREM2 protein corresponding to amino acid residues 124-153 of SEQ ID NO: 1; within amino acid residues 129-153 of SEQ ID NO: 1, or amino acid residues on a TREM2 protein corresponding to amino acid residues 129-153 of SEQ ID NO: 1; within amino acid residues 140-149 of SEQ ID NO: 1, or amino acid residues on a TREM2 protein corresponding to amino acid residues 140-149 of SEQ ID NO: 1; within amino acid residues 149-157 of SEQ ID NO: 1, or amino acid residues on a TREM2 protein corresponding to amino acid residues 149-157 of SEQ ID NO: 1; or within amino acid residues 153-162 of SEQ ID NO: 1, or amino acid residues on a TREM2 protein corresponding to amino acid residues 153-162 of SEQ ID NO: 1.

20. The method of any one of claims 1-19, wherein the antibody binds to one or more amino acid residues selected from the group consisting of D140, L141, W142, F143, P144, E151, D152, H154, E156, and H157 of SEQ ID NO: 1, or one or more amino acid residues on a mammalian TREM2 protein corresponding to an amino acid residue selected from the group consisting of D140, L141, W142, F143, P144, E151, D152, H154, E156, and H157 of SEQ ID NO: 1.

21. The method of any one of claims 1-20, wherein the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSQWMN (SEQ ID NO: 34), the HVR-H2 comprises the amino acid sequence RIYPGGGDTNYAGKFQG (SEQ ID NO: 35), the HVR-H3 comprises the amino acid sequence ARLLRNQPGESYAMDY (SEQ ID NO: 31), the HVR-L1 comprises the amino acid sequence RSSQSLVHSNRYTYLH (SEQ ID NO: 41), the HVR-L2 comprises the amino acid sequence KVSNRFS (SEQ ID NO: 33), and the HVR-L3 comprises the amino acid sequence SQSTRVPYT (SEQ ID NO: 32).

22. The method of any one of claims 1-21, wherein the antibody comprises a heavy chain variable region comprising the amino acid sequence of SEQ ID NO: 27 and a light chain variable region comprising the amino acid sequence of SEQ ID NO: 30

23. The method of any one of claims 1-20, wherein the antibody comprises a heavy chain variable region comprising an HVR-H1, HVR-H2, and HVR-H3 and a light chain variable region comprising an HVR-L1, HVR-L2, and HVR-L3, wherein the HVR-H1 comprises the amino acid sequence YAFSSDWMN (SEQ ID NO: 36), the HVR-H2 comprises the amino acid sequence RIYPGEGDTNYARKFHG (SEQ ID NO: 37), the HVR-H3 comprises the amino acid sequence ARLLRNKPGESYAMDY (SEQ ID NO: 38), the HVR-L1 comprises the amino acid sequence RTSQSLVHSNAYTYLH (SEQ ID NO: 39), the HVR-L2 comprises the amino acid sequence KVSNRVS (SEQ ID NO: 40), and the HVR-L3 comprises the amino acid sequence SQSTRVPYT (SEQ ID NO: 32).

24. The method of any one of claims 1-20 or 23, wherein the antibody comprises a heavy chain variable region comprising the amino acid sequence of SEQ ID NO: 28 and a light chain variable region comprising the amino acid sequence of SEQ ID NO: 29.

25. The method of any one of claims 1-24, wherein the antibody is a fragment and the fragment is an Fab, Fab′, Fab′-SH, F(ab′)2, Fv or scFv fragment.

26. The method of any one of claims 1-24, wherein the antibody is of the IgG class, the IgM class, or the IgA class.

27. The method of claim 26, wherein the antibody is of the IgG class and has an IgG1, IgG2, IgG3, or IgG4 isotype.

28. The method of claim 27, wherein the antibody has a human IgG1 isotype and comprises amino acid substitutions in the Fc region at the residue positions P331S and E430G, wherein the numbering of the residues is according to EU numbering.

29. The method of any one of claims 1-22, wherein the antibody comprises:
a. a heavy chain comprising the amino acid of SEQ ID NO: 43, and a light chain comprising the amino acid sequence of SEQ ID NO: 47; or
b. a heavy chain comprising the amino acid of SEQ ID NO: 44, and a light chain comprising the amino acid sequence of SEQ ID NO: 47.

30. The method of any one of claims 1-20 or 23-24, wherein the antibody comprises:
a. a heavy chain comprising the amino acid of SEQ ID NO: 45, and a light chain comprising the amino acid sequence of SEQ ID NO: 48; or
b. a heavy chain comprising the amino acid of SEQ ID NO: 46, and a light chain comprising the amino acid sequence of SEQ ID NO: 48.

31. The method of any one of claims 1-30, wherein the individual is a human.

32. The method of any of claims 1-31, wherein the CSF1R-deficient disease is adult-onset leukoencephalopathy with axonal spheroids and pigmented glia (ALSP).

33. The method of any of claims 1-31, wherein the CSF1R-deficient disease is pediatric-onset leukoencephalopathy.

34. The method of any one of claims 1-33, wherein the individual has a mutation in the CSF1R gene.

35. The method of claim 34, wherein the mutation is in the portion of the CSFR1 gene encoding the intracellular protein tyrosine kinase domain.

36. The method of claim 34, wherein the mutation is in any one of exons 11-21 of the CSFR1 gene.

37. The method of any one of claims 34-36, wherein the individual is heterozygous for the mutation in the CSFR1 gene.

38. The method of any one of claims 34-36, wherein the individual is homozygous for the mutation in the CSFR1 gene.

39. The method of any one of claims 1-38, wherein the individual has or is at risk for a disease characteristic selected from the group consisting of leukoencephalopathy, axonal damage, axonal spheroids, myelin damage, loss of myelin sheaths, gliosis, autofluorescent lipid-laden macrophages, and axon destruction.

40. The method of any one of claims 1-39, wherein the individual has or is at risk for a symptom selected from the group consisting of abnormality of the cerebral white matter, behavioral changes, dementia, parkinsonism, seizures, motor aphasia, agraphia, acalculia, apraxia, bradykinesia, slow movements, central nervous system demyelination, depressivity, depression, frontal lobe dementia, gliosis, hyperreflexia, increased reflexes, extensor plantar response, hemiparesis, quadriparesis, leukoencephalopathy, memory impairment, forgetfulness, memory loss, memory problems, poor memory, mutism, inability to speak, muteness, neuronal loss in central nervous system, loss of brain cells, postural instability, balance impairment, rapid progressivity, rigidity, muscle rigidity, shuffling gait, shuffled walk, pyramidial signs, spasticity, involuntary muscle stiffness, involuntary muscle contraction, involuntary muscle spasms, personality problems, executive dysfunction.

41. The method of any one of claims 1-40, wherein the individual has a disease selected from the group consisting of frontotemporal dementia (FTD), corticobasal syndrome (CBS), corticobasal degeneration (CBD), Alzheimer disease (AD), multiple sclerosis (MS), atypical cerebral autosomal dominant arteriopathy with subcortical infarcts and leukoencephalopathy (CADASIL), and Parkinson disease (PD).

42. A method of monitoring the treatment of an individual being administered an anti-TREM2 antibody comprising measuring the level of CSF1R in a sample from the individual before and after the individual has received one or more doses of an anti-TREM2 antibody.

43. The method of claim 42, further comprising a step of assessing the activity of the anti-TREM2 antibody in the individual based on the level of CSF1R in the sample.

44. The method of claim 42 or 43, wherein the sample is from the cerebrospinal fluid of the individual or the blood of the individual.


SEQUENCE LISTING (145 sequences)

SEQ ID NO: 1 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 2 - protein, 227 aa, Mus musculus
MGPLHQFLLLLITALSQALNTTVLQGMAGQSLRVSCTYDALKHWGRRKAWCRQLGEEGPCQRVVSTHGVWLLAFLKKRNGSTVIADDTLAGTVTITLKNLQAGDAGLYQCQSLRGREAEVLQKVLVEVLEDPLDDQDAGDLWVPEESSSFEGAQVEHSTSRNQETSFPPTSILLLLACVLLSKFLAASILWAVARGRQKPGTPVVRGLDCGQDAGHQLQILTGPGGT

SEQ ID NO: 3 - protein, 228 aa, Rattus rattus
MEPLHVFVLLLVTELSQALNTTVLQGVAGQSLRVSCTYDALRHWGRRKAWCRQLAEEGPCQRVVSTHGVWLLAFLRKQNGSTVITDDTLAGTVTITLRNLQAGDAGLYQCQSLRGREAEVLQKVVVEVLEDPLDDQDAGDLWVPEESESFEGAQVEHSTSSQVSSCGSPLTYHLPPKEPIRKDLLPTHFHSSPPGLCPPEQASYSQHPLGCGQGQAEAGDTCGQWARL

SEQ ID NO: 4 - protein, 260 aa, Macaca mulatta
MPDPLFSAVQGKDKILHKALCICPWPGKGGMEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPGLRDT

SEQ ID NO: 5 - protein, 230 aa, Macaca fascicularis
MEPLRLLILLFATELSGAHNTTVFQGVEGQSLQVSCPYDSMKHWGRRKAWCRQLGEKGPCQRVVSTHNLWLLSFLRRRNGSTAITDDTLGGTLTITLRNLQPHDAGFYQCQSLHGSEADTLRKVLVEVLADPLDHRDAGDLWVPGESESFEDAHVEHSISRSLLEGEIPFPPTSVLLLLACIFLIKILAASALWAAAWHGQKPGTHPPSEPDCGHDPGHQLQTLPGLRDT

SEQ ID NO: 6 - protein, 230 aa, Equus caballus
MEPLPLLILLSVAELSRGHNTTVFQGTAGRSLKVSCPYNSLMHWGRRKAWCRQLGEDGPCQQVVSTHSLWLLSFLKRRNGSTVITDDALGGILTITLRNLQAHDAGFYQCQSLHGGEADTLRKVLVEVLADPLDHQEPGDLWIPKESESFEDAQVEHSISRSLVEEEIPSLPTSILLLLACIFLSKLLAASAIWAAAWHGQKQETPPASEPDRGHDPGYQLHTLTGERDT

SEQ ID NO: 7 - protein, 233 aa, Sus scrofa
METLGLLLLLWVAELSRAHNTSVFQGTAGQSLRVSCSYNSLKHWGRRKAWCRQLSEEGLCQHVVSTHPTWLLSFLKRRNGSTAITDDALGGTLTITLRNLQAHDAGLYQCQSLHGSEADTLKKVLVEVLADPLESQSKSFQDVQMEHSISRNLSEESLFPPTSTLFLLACVFLSKLLVASALWAAAWHGHKQRTSPAGGLDCGRDPGDQDQTLTDELGESSDQDQTLTELRDT

SEQ ID NO: 8 - protein, 230 aa, Canis lupus familiaris
MEPLWLLILLAVTELSGAHNTTVFQGMAGRSLQVSCPYNSLKHWGRRKAWCRQVDKEGPCQRVVSTHRSWLLSFLKRWNGSTAIVDDALGGTLTITLRNLQAHDAGLYQCQSLYGDEADTLRKVLVEVLADPLDHLDPGDLWIPEESKGFEDAHVEPSVSRSLSEEEIPFPPTSILFLLACIFLSKFLAASALWAAAWRGQKLGTPQASELDCSCDPGYQLQTLTEPRDM

SEQ ID NO: 9 - protein, 26 aa, Artificial Sequence
  Synthetic Construct
QVQLVQSGAEVKKPGSSVKVSCKASG

SEQ ID NO: 10 - protein, 26 aa, Artificial Sequence
  Synthetic Construct
EVQLVQSGAEVKKPGSSVKVSCKASG

SEQ ID NO: 11 - protein, 26 aa, Artificial Sequence
  Synthetic Construct
QVQLVQSGAEVKKPGASVKVSCKASG

SEQ ID NO: 12 - protein, 14 aa, Artificial Sequence
  Synthetic Construct
WVRQAPGQGLEWMG

SEQ ID NO: 13 - protein, 14 aa, Artificial Sequence
  Synthetic Construct
WVRQAPGQRLEWIG

SEQ ID NO: 14 - protein, 30 aa, Artificial Sequence
  Synthetic Construct
RVTITADESTSTAYMELSSLRSEDTAVYYC

SEQ ID NO: 15 - protein, 30 aa, Artificial Sequence
  Synthetic Construct
RVTITADTSASTAYMELSSLRSEDTAVYYC

SEQ ID NO: 16 - protein, 11 aa, Artificial Sequence
  Synthetic Construct
WGQGTLVTVSS

SEQ ID NO: 17 - protein, 23 aa, Artificial Sequence
  Synthetic Construct
DVVMTQTPLSLSVTPGQPASISC

SEQ ID NO: 18 - protein, 23 aa, Artificial Sequence
  Synthetic Construct
GVVMTQTPLSLSVTPGQPASISC

SEQ ID NO: 19 - protein, 23 aa, Artificial Sequence
  Synthetic Construct
GVVMAQTPLSLSVTPGQPASISC

SEQ ID NO: 20 - protein, 23 aa, Artificial Sequence
  Synthetic Construct
DVVMTQSPDSLAVSLGERATINC

SEQ ID NO: 21 - protein, 15 aa, Artificial Sequence
  Synthetic Construct
WYLQKPGQSPQLLIY

SEQ ID NO: 22 - protein, 15 aa, Artificial Sequence
  Synthetic Construct
WYQQKPGQSPKLLIY

SEQ ID NO: 23 - protein, 32 aa, Artificial Sequence
  Synthetic Construct
GVPDRFSGSGSGTDFTLKISRVEAEDVGVYYC

SEQ ID NO: 24 - protein, 32 aa, Artificial Sequence
  Synthetic Construct
GVPDRFSGSGSGTDFTLTISSLQAEDVAVYYC

SEQ ID NO: 25 - protein, 10 aa, Artificial Sequence
  Synthetic Construct
FGQGTKLEIK

SEQ ID NO: 26 - protein, 10 aa, Artificial Sequence
  Synthetic Construct
FGGGTKVEIK

SEQ ID NO: 27 - protein, 123 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSS

SEQ ID NO: 28 - protein, 123 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSS

SEQ ID NO: 29 - protein, 112 aa, Artificial Sequence
  Synthetic Construct
DVVMTQTPLSLSVTPGQPASISCRTSQSLVHSNAYTYLHWYLQKPGQSPQLLIYKVSNRVSGVPDRFSGSGSGTDFTLKISRVEAEDVGVYYCSQSTRVPYTFGQGTKLEIK

SEQ ID NO: 30 - protein, 112 aa, Artificial Sequence
  Synthetic Construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIK

SEQ ID NO: 31 - protein, 16 aa, Artificial Sequence
  Synthetic Construct
ARLLRNQPGESYAMDY

SEQ ID NO: 32 - protein, 9 aa, Artificial Sequence
  Synthetic Construct
SQSTRVPYT

SEQ ID NO: 33 - protein, 7 aa, Artificial Sequence
  Synthetic Construct
KVSNRFS

SEQ ID NO: 34 - protein, 9 aa, Artificial Sequence
  Synthetic Construct
YAFSSQWMN

SEQ ID NO: 35 - protein, 17 aa, Artificial Sequence
  Synthetic Construct
RIYPGGGDTNYAGKFQG

SEQ ID NO: 36 - protein, 9 aa, Artificial Sequence
  Synthetic Construct
YAFSSDWMN

SEQ ID NO: 37 - protein, 17 aa, Artificial Sequence
  Synthetic Construct
RIYPGEGDTNYARKFHG

SEQ ID NO: 38 - protein, 16 aa, Artificial Sequence
  Synthetic Construct
ARLLRNKPGESYAMDY

SEQ ID NO: 39 - protein, 16 aa, Artificial Sequence
  Synthetic Construct
RTSQSLVHSNAYTYLH

SEQ ID NO: 40 - protein, 7 aa, Artificial Sequence
  Synthetic Construct
KVSNRVS

SEQ ID NO: 41 - protein, 16 aa, Artificial Sequence
  Synthetic Construct
RSSQSLVHSNRYTYLH

SEQ ID NO: 42 - protein, 110 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 43 - protein, 453 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 44 - protein, 452 aa, Artificial Sequence
  Synthetic Construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPG

SEQ ID NO: 45 - protein, 453 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 452 aa, Artificial Sequence
  Synthetic Construct
QVQLVQSGAEVKKPGSSVKVSCKASGYAFSSDWMNWVRQAPGQGLEWMGRIYPGEGDTNYARKFHGRVTITADESTSTAYMELSSLRSEDTAVYYCARLLRNKPGESYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 219 aa, Artificial Sequence
  Synthetic Construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 219 aa, Artificial Sequence
  Synthetic Construct
DVVMTQTPLSLSVTPGQPASISCRTSQSLVHSNAYTYLHWYLQKPGQSPQLLIYKVSNRVSGVPDRFSGSGSGTDFTLKISRVEAEDVGVYYCSQSTRVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 19 aa, Artificial Sequence
  Synthetic Construct
  VARIANT 1: Xaa = Asp or Glu
  VARIANT 2, 3: Xaa = Any Amino Acid, and up to two can be present or absent
  VARIANT 5, 6: Xaa = Any Amino Acid
  VARIANT 7: Xaa = Leu or Ile
  VARIANT 8, 9, 10, 11, 12, 13, 14, 15: Xaa = Any Amino Acid, and up to two can be present or absent
  VARIANT 17, 18: Xaa = Any Amino Acid
  VARIANT 19: Xaa = Leu or Ile
XXXYXXXXXXXXXXXYXXX

SEQ ID NO: 50 - protein, 11 aa, Artificial Sequence
  Synthetic Construct
GYSITSDYAWN

SEQ ID NO: 51 - protein, 16 aa, Artificial Sequence
  Synthetic Construct
YINYSGRTIYNPSLKS

SEQ ID NO: 52 - protein, 11 aa, Artificial Sequence
  Synthetic Construct
ARWNGNYGFAY

SEQ ID NO: 53 - protein, 16 aa, Artificial Sequence
  Synthetic Construct
RSSQSLVHINGNTYLH

SEQ ID NO: 54 - protein, 7 aa, Artificial Sequence
  Synthetic Construct
KVSNRFS

SEQ ID NO: 55 - protein, 9 aa, Artificial Sequence
  Synthetic Construct
SQTTHALFT

SEQ ID NO: 56 - protein, 118 aa, Artificial Sequence
  Synthetic Construct
DVQLQESGPGLVKPSQSLSLTCTVTGYSITSDYAWNWIRQFPGNKLEWMGYINYSGRTIYNPSLKSRISITRDTSKNHFFLQLISVTTEDTATYYCARWNGNYGFAYWGQGTLVTVSA

SEQ ID NO: 57 - protein, 111 aa, Artificial Sequence
  Synthetic Construct
DWMTQNPLSLPVSLGDQASISCRSSQSLVHINGNTYLHWYLQKPGQSPKLLIYKVSNRFSGVPDRFSGSGSGTDFTLKISRVEAEDLGVYFCSQTTHALFTFGSGTKLEIK

SEQ ID NO: 58 - protein, 7 aa, Artificial Sequence
  Synthetic Construct
GYTFTSY

SEQ ID NO: 59 - protein, 15 aa, Artificial Sequence
  Synthetic Construct
IGRSDPTTGGTNYNE

SEQ ID NO: 60 - protein, 9 aa, Artificial Sequence
  Synthetic Construct
VRTSGTGDY

SEQ ID NO: 61 - protein, 16 aa, Artificial Sequence
  Synthetic Construct
RSSQSLVHNNGNTFLH

SEQ ID NO: 62 - protein, 6 aa, Artificial Sequence
  Synthetic Construct
VSNRFS

SEQ ID NO: 63 - protein, 9 aa, Artificial Sequence
  Synthetic Construct
SQTTHVPPT

SEQ ID NO: 64 - protein, 140 aa, Artificial Sequence
  Synthetic Construct
QVQLQQPGAELVKPGASVKLSCKASGYTFTSYWMHWVKQSPGRGLEWIGRSDPTTGGTNYNEKFKTKATLTVDKPSSTAYMQLSSLTSDDSAVYYCVRTSGTGDYWGQGTSLTVSSAKTTAPSVYPLAPVCGGTTGSSVT

SEQ ID NO: 65 - protein, 140 aa, Artificial Sequence
  Synthetic Construct
DVVMTQTPLSLPVSLGDQASISCRSSQSLVHNNGNTFLHWYLQKPGQSPKLLIYKVSNRFSGVPDRFSGSGSGTDFTLKISRVEAEDLGVYFCSQTTHVPPTFGGGTKLEIKRADAAPTVSIFPPSSEQLTSGGASVVCF

SEQ ID NO: 66 - protein, 8 aa, Artificial Sequence
  Synthetic Construct
GFTFTDFY

SEQ ID NO: 67 - protein, 10 aa, Artificial Sequence
  Synthetic Construct
IRNKANGYTT

SEQ ID NO: 68 - protein, 15 aa, Artificial Sequence
  Synthetic Construct
ARIGINNGGSLDYWG

SEQ ID NO: 69 - protein, 12 aa, Artificial Sequence
  Synthetic Construct
QSLLYSENNQDY

SEQ ID NO: 70 - protein, 3 aa, Artificial Sequence
  Synthetic Construct
GAS

SEQ ID NO: 71 - protein, 9 aa, Artificial Sequence
  Synthetic Construct
EQTYSYPYT

SEQ ID NO: 72 - protein, 121 aa, Artificial Sequence
  Synthetic Construct
EVKLLESGGGLVQPGGSMRLSCAASGFTFTDFYMNWIRQPAGKAPEWLGLIRNKANGYTTEYNPSVKGRFTISRDNTQNMLYLQMNTLREDTATYYCARIGINNGGSLDYWGQGVMVTVSS

SEQ ID NO: 73 - protein, 113 aa, Artificial Sequence
  Synthetic Construct
DILINQSPASLTVSAGEKVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 74 - protein, 8 aa, Artificial Sequence
  Synthetic Construct
GFTFTDFY

SEQ ID NO: 75 - protein, 10 aa, Artificial Sequence
  Synthetic Construct
IRNKTKGYTT

SEQ ID NO: 76 - protein, 15 aa, Artificial Sequence
  Synthetic Construct
ARIGVNNGGSLDYWG

SEQ ID NO: 77 - protein, 12 aa, Artificial Sequence
  Synthetic Construct
QSLLYSENNQDY

SEQ ID NO: 78 - protein, 3 aa, Artificial Sequence
  Synthetic Construct
GAS

SEQ ID NO: 79 - protein, 9 aa, Artificial Sequence
  Synthetic Construct
EQTYSYPYT

SEQ ID NO: 80 - protein, 122 aa, Artificial Sequence
  Synthetic Construct
EVKLLEFGGGLVQPGGSMRLSCAASGFTFTDFYMNWIRQPAGRAPEWLGLIRNKTKGYTTEYNRSVKGRFTISRDNTQNMLYLQMNSLRPEDTATYYCARIGVNNGGSLDYWGQGVMVTVSS

SEQ ID NO: 81 - protein, 113 aa, Artificial Sequence
  Synthetic Construct
DILIIQSPASLTVSAGARVTMSCKSSQSLLYSENNQDYLAWYQQKPGQFPKLLIYGASNRHTGVPDRFTGSGSGTDFTLTISSVQAEDLADYYCEQTYSYPYTFGAGTKLELK

SEQ ID NO: 82 - protein, 8 aa, Artificial Sequence
  Synthetic Construct
GFTFTDFY

SEQ ID NO: 83 - protein, 10 aa, Artificial Sequence
  Synthetic Construct
IRNKANGYTT

SEQ ID NO: 84 - protein, 15 aa, Artificial Sequence
  Synthetic Construct
ARIGINNGGSLDYWG

SEQ ID NO: 85 - protein, 12 aa, Artificial Sequence
  Synthetic Construct
QSLLYSEKNQDY

SEQ ID NO: 86 - protein, 3 aa, Artificial Sequence
  Synthetic Construct
GAS

SEQ ID NO: 87 - protein, 9 aa, Artificial Sequence
  Synthetic Construct
EQTYSYPYT

SEQ ID NO: 88 - protein, 122 aa, Artificial Sequence
  Synthetic Construct
EVKLLESGGGLVQPGGSMRLSCAASGFTFTDFYMNWIRQPAGKAPEWLGLIRNKANGYTTVYNPSVKGRFTISRDNTQNMLYLQMNTLRGEDTATYYCARIGINNGGSLDYWGQGVMVTVSS

SEQ ID NO: 89 - protein, 113 aa, Artificial Sequence
  Synthetic Construct
DILINQSPASLTVSTGEKVTMSCRSSQSLLYSEKNQDYLAWYQQKPGQFPKLLIYGASYRHTGVPDRFTGSGSGTDFTLTISSVQAEDLADYYCEQTYSYPYTFGAGTKLELK

SEQ ID NO: 90 - protein, 8 aa, Artificial Sequence
  Synthetic Construct
GFTFTDFY

SEQ ID NO: 91 - protein, 10 aa, Artificial Sequence
  Synthetic Construct
IRNKANGYTT

SEQ ID NO: 92 - protein, 15 aa, Artificial Sequence
  Synthetic Construct
ARIGINNGGSLDYWG

SEQ ID NO: 93 - protein, 12 aa, Artificial Sequence
  Synthetic Construct
QSLLYSEKNQDY

SEQ ID NO: 94 - protein, 3 aa, Artificial Sequence
  Synthetic Construct
GAS

SEQ ID NO: 95 - protein, 9 aa, Artificial Sequence
  Synthetic Construct
EQTYSYPYT

SEQ ID NO: 96 - protein, 122 aa, Artificial Sequence
  Synthetic Construct
EVKLLESGGGLVQPGGSMRLSCAASGFTFTDFYMNWIRQPAGKAPEWLGLIRNKANGYTTQYNPSVKGRFTISRDNTQNMLYLQMNTLRGEDTATYYCARIGINNGGSLDYWGQGVMVTVSS

SEQ ID NO: 97 - protein, 113 aa, Artificial Sequence
  Synthetic Construct
DILINQSPASLTVSAGEKVTMSCKSSQSLLYSEKNQDYLAWYQQKPGQSPKLLMYGASYRHTGVPDRFTGSGSGTDFTLTISSVQAEDLADYYCEQTYSYPYTFGAGTKLELK

SEQ ID NO: 98 - protein, 8 aa, Artificial Sequence
  Synthetic Construct
GFTFTDFY

SEQ ID NO: 99 - protein, 10 aa, Artificial Sequence
  Synthetic Construct
IRNKTKGYTT

SEQ ID NO: 100 - protein, 15 aa, Artificial Sequence
  Synthetic Construct
ARIGVNNGGSLDYWG

SEQ ID NO: 101 - protein, 12 aa, Artificial Sequence
  Synthetic Construct
QSLLYSENNQDY

SEQ ID NO: 102 - protein, 3 aa, Artificial Sequence
  Synthetic Construct
GAS

SEQ ID NO: 103 - protein, 9 aa, Artificial Sequence
  Synthetic Construct
EQTYSYPYT

SEQ ID NO: 104 - protein, 122 aa, Artificial Sequence
  Synthetic Construct
EVKLLESGGGLVQPGGSMRLSCAASGFTFTDFYMNWIRQPAGKAPEWLGLIRNKTKGYTTEYNTSVKGRFTISRDNTQNMLYLQMNSLRPEDTATYYCARIGVNNGGSLDYWGQGVMVTVSS

SEQ ID NO: 105 - protein, 113 aa, Artificial Sequence; identical to SEQ ID NO: 81
  Synthetic Construct

SEQ ID NO: 106 - protein, 8 aa, Artificial Sequence
  Synthetic Construct
GFTFTDFY

SEQ ID NO: 107 - protein, 10 aa, Artificial Sequence
  Synthetic Construct
IRNKANGYTT

SEQ ID NO: 108 - protein, 15 aa, Artificial Sequence
  Synthetic Construct
ARIGINNGGSLDYWG

SEQ ID NO: 109 - protein, 12 aa, Artificial Sequence
  Synthetic Construct
QSLLYSEKNQDY

SEQ ID NO: 110 - protein, 3 aa, Artificial Sequence
  Synthetic Construct
GAS

SEQ ID NO: 111 - protein, 9 aa, Artificial Sequence
  Synthetic Construct
EQTYSYPYT

SEQ ID NO: 112 - protein, 122 aa, Artificial Sequence; identical to SEQ ID NO: 96
  Synthetic Construct

SEQ ID NO: 113 - protein, 113 aa, Artificial Sequence; identical to SEQ ID NO: 97
  Synthetic Construct

SEQ ID NO: 114 - protein, 8 aa, Artificial Sequence
  Synthetic Construct
GFTFTDFY

SEQ ID NO: 115 - protein, 10 aa, Artificial Sequence
  Synthetic Construct
IRNKTKGYTT

SEQ ID NO: 116 - protein, 15 aa, Artificial Sequence
  Synthetic Construct
ARIGTNNGGSLDYWG

SEQ ID NO: 117 - protein, 12 aa, Artificial Sequence
  Synthetic Construct
QSLLYSENNQDY

SEQ ID NO: 118 - protein, 3 aa, Artificial Sequence
  Synthetic Construct
GAS

SEQ ID NO: 119 - protein, 9 aa, Artificial Sequence
  Synthetic Construct
EQTYSYPYT

SEQ ID NO: 120 - protein, 122 aa, Artificial Sequence
  Synthetic Construct
EVKLLESGGGLVQPGGSMRLSCAASGFTFTDFYMNWIRQPAGETPEWLGLIRNKTKGYTTEYNPSVKGRFTISRDNTQNMLYLQMNSLRPEDTATYYCARIGTNNGGSLDYWGQGVMVTVSS

SEQ ID NO: 121 - protein, 113 aa, Artificial Sequence; identical to SEQ ID NO: 81
  Synthetic Construct

SEQ ID NO: 122 - protein, 8 aa, Artificial Sequence
  Synthetic Construct
GFTFTDFY

SEQ ID NO: 123 - protein, 10 aa, Artificial Sequence
  Synthetic Construct
IRNKVNGYRT

SEQ ID NO: 124 - protein, 15 aa, Artificial Sequence
  Synthetic Construct
ARIGINNGGSLDYWG

SEQ ID NO: 125 - protein, 12 aa, Artificial Sequence
  Synthetic Construct
QSLLYSENNQDY

SEQ ID NO: 126 - protein, 3 aa, Artificial Sequence
  Synthetic Construct
GAS

SEQ ID NO: 127 - protein, 9 aa, Artificial Sequence
  Synthetic Construct
EQTYSYPYT

SEQ ID NO: 128 - protein, 122 aa, Artificial Sequence
  Synthetic Construct
EVKLLESGGGLVQPGGSMRLSCVVSGFTFTDFYMNWIRQAAGKAPEWLGLIRNKVNGYRTEYNPSVKGRFTISRDNIQNMLYLQMNTLRAEDTATYYCARIGINNGGSLDYWGQGVMVTVSS

SEQ ID NO: 129 - protein, 113 aa, Artificial Sequence; identical to SEQ ID NO: 73
  Synthetic Construct

SEQ ID NO: 130 - protein, 8 aa, Artificial Sequence
  Synthetic Construct
GFTFTDFY

SEQ ID NO: 131 - protein, 10 aa, Artificial Sequence
  Synthetic Construct
IRNKAYGYTT

SEQ ID NO: 132 - protein, 15 aa, Artificial Sequence
  Synthetic Construct
ARIGINYGGSLDYWG

SEQ ID NO: 133 - protein, 12 aa, Artificial Sequence
  Synthetic Construct
QSLLYSESNQDY

SEQ ID NO: 134 - protein, 3 aa, Artificial Sequence
  Synthetic Construct
GAS

SEQ ID NO: 135 - protein, 9 aa, Artificial Sequence
  Synthetic Construct
EQTYSYPYT

SEQ ID NO: 136 - protein, 122 aa, Artificial Sequence
  Synthetic Construct
EVKLLESGGGLVQPGGSMRLSCAASGFTFTDFYMNWIRQPAGKAPEWLGLIRNKAYGYTTEYNPSVKGRFTISRDNTQDMLYLQMNTLRAEDTATYYCARIGINYGGSLDYWGQGVMVTVSS

SEQ ID NO: 137 - protein, 113 aa, Artificial Sequence
  Synthetic Construct
DILINQSPASLTVSAGEKVTVSCKSSQSLLYSESNQDYLAWYQQKPGQFPKLLIYGASYRHTGVPDRFTGSGSGTDFTLTISSVQAEDLAHYYCEQTYSYPYTFGAGTKLELK

SEQ ID NO: 138 - protein, 8 aa, Artificial Sequence
  Synthetic Construct
GFTFTDFY

SEQ ID NO: 139 - protein, 10 aa, Artificial Sequence
  Synthetic Construct
IRNKANGFTT

SEQ ID NO: 140 - protein, 15 aa, Artificial Sequence
  Synthetic Construct
ARIGINNGGSLDYWG

SEQ ID NO: 141 - protein, 12 aa, Artificial Sequence
  Synthetic Construct
QSLLYSENKQDY

SEQ ID NO: 142 - protein, 3 aa, Artificial Sequence
  Synthetic Construct
GAS

SEQ ID NO: 143 - protein, 9 aa, Artificial Sequence
  Synthetic Construct
EQTYSYPYT

SEQ ID NO: 144 - protein, 122 aa, Artificial Sequence
  Synthetic Construct
EVKLLESGGGLVQPGGSLRLSCVASGFTFTDFYMNWIRQPAGKAPEWLGLIRNKANGFTTEYNPSVKGRFTISRDNTQHMLYLQMNTLRAEDTATYYCARIGINNGGSLDYWGQGVMVTVSS

SEQ ID NO: 145 - protein, 113 aa, Artificial Sequence
  Synthetic Construct
DILINQSPASLTVSTGEKVTMSCKSSQSLLYSENKQDYLAWYQQKPGQFPKLLIYGASNRHTGVPDRFTGSGSGTDFTLTINIVQAEDLADYYCEQTYSYPYTFGAGTKLELK